SEQUENCE LISTING (29 sequences)

SEQ ID NO: 1 - DNA, 86 bp, synthetic construct
  source 1..86: mol_type = other DNA
gtcagaaaaaacgggtttcctggttggctcggagagcatcaggcgatgctctccgttccaacaaggaaaacagacagtaactcaga

SEQ ID NO: 2 - RNA, 69 nt, synthetic construct
  source 1..69: mol_type = other RNA
atgcgcacccttagcgagaggtttatcattaaggtcaacctctggatgttgtttcggcatcctgcattg

SEQ ID NO: 3 - DNA, 57 bp, synthetic construct
  source 1..57: mol_type = other DNA
gtcagaaaaaacgggttgtcgccagtctgactggcgacaaacagcttgtaactcaga

SEQ ID NO: 4 - DNA, 55 bp, synthetic construct
  source 1..55: mol_type = other DNA
gtcagaaaaaacgggtggaggttgctgcaacctctccattttcttgtaactcaga

SEQ ID NO: 5 - DNA, 57 bp, synthetic construct; identical to SEQ ID NO: 3
  source 1..57: mol_type = other DNA

SEQ ID NO: 6 - DNA, 57 bp, synthetic construct; identical to SEQ ID NO: 3
  source 1..57: mol_type = other DNA

SEQ ID NO: 7 - DNA, 56 bp, synthetic construct
  source 1..56: mol_type = other DNA
gtcagaaaaaacgggttgtcgccagtctgactggcgacaaacagcttgtaactcag

SEQ ID NO: 8 - DNA, 88 bp, synthetic construct
  source 1..88: mol_type = other DNA
gtcagaaaaaacgggtttcctggttggctcggagagcatcaggcgatgctctctccgttccaacaaggaaaacagacagtaactcaga

SEQ ID NO: 9 - DNA, 87 bp, synthetic construct
  source 1..87: mol_type = other DNA
gtcagaaaaaacgggtttcctggttggctcggagagcatcaggcgatgctctctccgttccaacaaggaaaacagacagtaactcag

SEQ ID NO: 10 - DNA, 83 bp, synthetic construct
  source 1..83: mol_type = other DNA
ttgaagccgcggaacaaactttttgatccgcaacctactggattgcggctcaaaaagtttgttccgcaactgtaaatgtaatc

SEQ ID NO: 11 - DNA, 79 bp, synthetic construct
  source 1..79: mol_type = other DNA
agccgcggaacaaactttttgatccgcaacctactggattgcggctcaaaaagtttgttccgcaactgtaaatgtaatc

SEQ ID NO: 12 - DNA, 169 bp, synthetic construct
  source 1..169: mol_type = other DNA
tgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgca

SEQ ID NO: 13 - DNA, 199 bp, synthetic construct
  source 1..199: mol_type = other DNA
tgataagattccgtatgcgcacccttagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgtaagggtgcgcatacggaatcttatca

SEQ ID NO: 14 - DNA, 86 bp, synthetic construct; identical to SEQ ID NO: 1
  source 1..86: mol_type = other DNA

SEQ ID NO: 15 - DNA, 169 bp, synthetic construct; identical to SEQ ID NO: 12
  source 1..169: mol_type = other DNA

SEQ ID NO: 16 - DNA, 97 bp, synthetic construct
  source 1..97: mol_type = other DNA
tgcgcacccttagcgagaggtttatcattaaggtcaacctctggatgttgtttcggcatcctgcattgaatctgagttacaagctgtttgtcgccag

SEQ ID NO: 17 - DNA, 72 bp, synthetic construct
  source 1..72: mol_type = other DNA
aatctgagttacaagctgtttgtcgccagtcagactggcgacaacccgttttttctgacgtaagggtgcgca

SEQ ID NO: 18 - DNA, 140 bp, synthetic construct
  source 1..140: mol_type = other DNA
tgcgcacccttagcgagaggtttatcattaaggtcaacctctggatgttgtttcggcatcctgcattgaatctgagttacaagctgtttgtcgccagtcagactggcgacaacccgttttttctgacgtaagggtgcgca

SEQ ID NO: 19 - DNA, 140 bp, synthetic construct; identical to SEQ ID NO: 18
  source 1..140: mol_type = other DNA

SEQ ID NO: 20 - DNA, 123 bp, synthetic construct
  source 1..123: mol_type = other DNA
tggacaatattgaatggagtctgatcaaccttcacaccgatctagaatcggaatctgagttacaagctgtttgtcgccagtcagactggcgacaacccgttttttctgacgtaagggtgcgca

SEQ ID NO: 21 - DNA, 199 bp, synthetic construct; identical to SEQ ID NO: 13
  source 1..199: mol_type = other DNA

SEQ ID NO: 22 - DNA, 170 bp, synthetic construct
  source 1..170: mol_type = other DNA
tgataagattccgtatgcgcacccttagcgagaggtttatcattaaggtcaacctctggatgttgtttcggcatcctgcattgaatctgagttacaagctgtttgtcgccagtcagactggcgacaacccgttttttctgacgtaagggtgcgcatacggaatcttatca

SEQ ID NO: 23 - DNA, 149 bp, synthetic construct
  source 1..149: mol_type = other DNA
tagcgagaggtttatcattaaggtcaacctctggatgttgtttcggcatcctgcattgaatctgagttactgtctgttttccttgttggaacggagagcatcgcctgatgctctccgagccaaccaggaaacccgttttttctgacgta

SEQ ID NO: 24 - DNA, 147 bp, synthetic construct
  source 1..147: mol_type = other DNA
tgcgcacccttagcgagaggtttatcattaaggtcaacctctggatgttgtttcggcatcctgcattgaatctgagttactgtctgttttccttgttggaagcctagccaaccaggaaacccgttttttctgacgtaagggtgcgca

SEQ ID NO: 25 - DNA, 123 bp, synthetic construct
  source 1..123: mol_type = other DNA
tgcgcacccttagcgagaggtttatcattaaggtcaacctctggatgttgtttcggcatcctgcattgaatctgagttactgtctgtttgcctaacccgttttttctgacgtaagggtgcgca

SEQ ID NO: 26 - DNA, 169 bp, synthetic construct
  source 1..169: mol_type = other DNA
tgcgcacccttagcgagaggtttatcattaaggtcaacctctggatgttgtttcggcatcctgcattgaatctgagttactgtctgttttcgaacatggaacggagagcatcgcctgatgctctccgagccaaccaggaaacccgttttttctgacgtaagggtgcgca

SEQ ID NO: 27 - DNA, 169 bp, synthetic construct
  source 1..169: mol_type = other DNA
tgcgcacccttagcgagaggtttatcattaaggtcaacctctggatgttgtttcggcatcctgcattgaatctgagttactgtctgttttcgaacatggaacggagagcatcgcctgatgctctccgagccatggtcgaaacccgttttttctgacgtaagggtgcgca

SEQ ID NO: 28 - DNA, 169 bp, synthetic construct
  source 1..169: mol_type = other DNA
tgcgcacccttagcgagaggtttatcattaaggtcaacctctggatgttgtttcggcatcctgcattgaatctgagttactgtctgttttccttgttggaacgctctccatcgcctgatgctctccgagccaaccaggaaacccgttttttctgacgtaagggtgcgca

SEQ ID NO: 29 - DNA, 12 bp, synthetic construct
  source 1..12: mol_type = other DNA
cccccccccccc

(19) United States
(12) Patent Application Publication
(10) Pub. No.: US 2025/0188442 A1
(43) Pub. Date: Jun. 12, 2025

(54) MODIFIED BACTERIAL RETROELEMENT WITH ENHANCED DNA PRODUCTION

(71) Applicants: The J. David Gladstone Institutes, a testamentary trust established under the will of J. David, San Francisco, CA (US); President and Fellows of Harvard College, Cambridge, MA (US)

(72) Inventor: Seth Shipman, San Francisco, CA (US)

(21) Appl. No.: 18/949,679

(22) Filed: Nov. 15, 2024

Related U.S. Application Data
(63) Continuation of application No. 17/639,043, filed on Feb. 28, 2022, now abandoned.
(60) Provisional application No. 62/899,625, filed on Sep. 12, 2019.

(51) Int. Cl. C12N 15/10; C12N 9/12; C12N 9/22; C12N 15/63; C12Q 1/48

(52) U.S. Cl. CPC C12N 15/102; C12N 9/1276; C12N 9/22; C12N 15/63; C12Q 1/48; C12N 2310/20; C12N 2800/80; C12Q 2563/179

(57) ABSTRACT

Engineered retrons, modified to enhance production of multicopy single-stranded DNA (msDNA), are provided. In addition, vector systems encoding such engineered retrons and methods of using engineered retrons and vector systems encoding them in various applications such as CRISPR/Cas-mediated genome editing, recombineering, cellular barcoding, and molecular recording are also disclosed.

### PRIORITY APPLICATION

This application is a continuation of U.S. patent application Ser. No. 17/639,043, filed Feb. 28, 2022, which application is a U.S. National Stage Filing under 35 U.S.C. 371 from International Patent Application Serial No.

PCT/US2020/050323, filed Sep. 11, 2020, which application claims benefit of priority to the filing date of U.S. Provisional Application Ser. No. 62/899,625, filed Sep. 12, 2019, the contents of which are specifically incorporated herein by reference in their entireties.

### INCORPORATION BY REFERENCE OF SEQUENCE LISTING

This application contains a Sequence Listing which has been submitted electronically in ST26 format and hereby incorporated by reference in its entirety. Said ST26 file, created on Nov. 15, 2024, is named 3730112US3.xml and is 29,247 bytes in size.

### BACKGROUND

Retrons are reverse transcribed elements found in nearly all myxobacteria (Dhundale et al. Journal of Bacteriology 164, 914-917 (1985)) and sparsely in E. coli (Lampson et al. Science 243, 1033-1038 (1989)), V. cholerae (Inouye et al. Microbiology and Immunology 55, 510-513), and other bacteria. The retron operon encodes an RNA primer (multicopy single-stranded RNA, msr), an RNA sequence to be reverse-transcribed (multicopy single-stranded DNA, msd), and a reverse transcriptase, in that order. The retron transcript folds up upon itself and is partially reverse-transcribed to generate a single stranded DNA (ssDNA) of about 80 bases.

Although the retron-derived DNA is single stranded, it contains a hairpin of double-stranded DNA. Multiple retron ssDNAs can also complement each other to form larger double-stranded elements. Retron variants have different DNA lengths and base content, but broadly share this overall format.

The ssDNA generated by the retron has been used for genome engineering in two contexts: bacterial, with the λ Red Beta recombinase for recombineering (Farzadfard et al. Science 346, 1256272, (2014)); and eukaryotic, as a homology-directed repair (HDR) template for Cas9 editing (Sharon et al. Cell 175, 544-557.e516, (2018)) in yeast. Despite tremendous promise, these applications suffered from lower-than-expected efficiency and context-restriction, which likely stem from elements in the endogenous form of the retron. These include (1) a branched structure with a phosphodiester bond linking the 5′ end of the ssDNA to a 2′ hydroxyl of the msr RNA, (2) invariant flanking regions that may be required for retron reverse transcription, but are not part of the repair template, (3) limited total length, and (4) a native poly T stretch that functions as a terminator for Pol III transcription.

### SUMMARY

Engineered retrons, modified to enhance production of multicopy single-stranded DNA (msDNA), are provided that solve many of the existing problems relating to efficiency and low copy numbers. Also described herein are vector systems encoding such engineered retrons and methods of using engineered retrons and vector systems in various applications such as CRISPR/Cas-mediated genome editing, recombineering, cellular barcoding, and molecular recording.

In one aspect, an engineered retron is provided, the engineered retron comprising: a) a pre-msr sequence; b) an msr gene encoding multicopy single-stranded RNA (msRNA); c) an msd gene encoding multicopy single-stranded DNA (msDNA); d) a post-msd sequence comprising a self-complementary region having sequence complementarity to the pre-msr sequence, wherein the self-complementary region has a length of at least 1 to 50 nucleotides longer than a wild-type complementary region such that the engineered retron is capable of enhanced production of the msDNA; and e) a ret gene encoding a reverse transcriptase.

The self-complementary region is formed by hydrogen bonding between the 3′ and 5′ ends of the neRNA. In certain embodiments, the complementary region has a length that is at least 1, at least 2, at least 4, at least 6, at least 8, at least 10, at least 12, at least 14, at least 16, at least 18, at least 20, at least 30, at least 40, or at least 50 nucleotides longer than the wild-type complementary region. For example, the self-complementary region may have a length ranging from 1 to 50 nucleotides longer than the wild-type complementary region, including any length within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47, 48, 49, or 50 nucleotides longer. In certain embodiments, the self-complementary region has a length ranging from 1 to 16 nucleotides longer than the wild-type complementary region.

In certain embodiments, the msr gene and the msd gene are provided in a trans arrangement or a cis arrangement. In some embodiments, the ret gene is provided in a trans arrangement with respect to the msr gene and/or the msd gene.

In certain embodiments, the msr gene, msd gene, and ret gene are derived from a bacterial retron including, without limitation, a myxobacteria retron (e.g., Mx65, Mx162), an Escherichia coli retron (e.g., E67, Ec73, EC83, EC86, EC107), a Salmonella enterica retron (e.g., msDNA-St85) and a Vibrio cholerae retron (e.g., Vc81, Vc95, Vc137).

In certain embodiments, the engineered retron further comprises a heterologous sequence of interest. The heterologous sequence may be inserted, for example, into the msr gene or the msd gene. For example, the heterologous sequence can be inserted into the loop of the msd stem loop. In some embodiments, the heterologous sequence encodes a polypeptide or peptide. In other embodiments, the heterologous sequence encodes a donor polynucleotide comprising a 5′ homology arm that hybridizes to a 5′ genomic target sequence and a 3′ homology arm that hybridizes to a 3′ genomic target sequence flanking a nucleotide sequence comprising an intended edit to be integrated at a genomic target locus by homology directed repair (HDR) or recombineering. In yet other embodiments, the heterologous sequence comprises a CRISPR protospacer DNA sequence. In one embodiment, the CRISPR protospacer DNA sequence comprises a modified “AAG” protospacer adjacent motif (PAM).

In certain embodiments, the engineered retron further comprises a barcode sequence. The barcode sequence may be located, for example, in a hairpin loop of the msDNA.

In another aspect, a vector system is provided, the vector system comprising one or more vectors comprising an engineered retron described herein. In certain embodiments, the msr gene and the msd gene are provided by the same vector or different vectors. In some embodiments, the msr gene, the msd gene, and the ret gene are provided by the same vector, wherein the vector comprises a promoter operably linked to the msr gene and the msd gene. In some embodiments, the promoter is further operably linked to the ret gene. In other embodiments, the vector further comprises a second promoter operably linked to the ret gene. In certain embodiments, the msr gene, the msd gene, and the ret gene are provided by different vectors.

In certain embodiments, one or more of the vectors of the vector system are viral vectors or nonviral vectors (e.g., plasmids).

In certain embodiments, the vector system comprises an engineered retron comprising a heterologous sequence encoding a donor polynucleotide comprising a 5′ homology arm that hybridizes to a 5′ genomic target sequence and a 3′ homology arm that hybridizes to a 3′ genomic target sequence flanking the donor polynucleotide sequence. The donor polynucleotide sequence can replace or edit a genomic target locus, for example, by homology directed repair (HDR) or recombineering.

In certain embodiments, the vector system further comprises a vector encoding an RNA-guided nuclease. Exemplary RNA-guided nucleases include, without limitation, Cas nucleases (e.g., Cas9, Cpf1) and engineered RNA-guided FokI-nuclease

In certain embodiments, the vector system further comprises a vector encoding bacteriophage recombination proteins for recombineering. In some embodiments, the vector is a replication-defective prophage encoding the bacteriophage recombination proteins.

In certain embodiments, the vector system comprises an engineered retron comprising a heterologous sequence encoding a CRISPR protospacer DNA sequence. In some embodiments, the vector system further comprises a vector encoding a Cas1 or Cas2 protein. In some embodiments, the vector system further comprises a vector comprising a CRISPR array sequence.

In another aspect, an isolated host cell is provided, the host cell comprising an engineered retron or a vector system described herein.

In certain embodiments, the bost cell is a prokaryotic, archeon, or eukaryotic host cell. For example, the host cell may be a bacterial, protist, fungal, animal, or plant host cell. In some embodiments, the host cell is a mammalian host cell. The host cell may be a human or nonhuman mammalian host cell. In other embodiments, the host cell is an artificial cell or a genetically modified cell.

In another aspect, a kit comprising an engineered retron, described herein, or a vector system or a host cell comprising such an engineered retron is provided. In some embodiments, the kit further comprises instructions on methods of using the engineered retron.

In another aspect, a method of genetically modifying a cell is provided. In some cases, the method includes transfecting a cell with an engineered retron. For example, the method can include: a) transfecting a cell with an engineered retron comprising a heterologous sequence encoding a donor polynucleotide comprising a 5′ homology arm that hybridizes to a 5′ genomic target sequence and a 3′ homology arm that hybridizes to a 3′ genomic target sequence flanking a nucleotide sequence comprising an intended edit to be integrated at a genomic target locus by homology directed repair (HDR); and b) introducing an RNA-guided nuclease and guide RNA into the cell, wherein the RNA-guided nuclease forms a complex with the guide RNA, said guide RNAs directing the complex to the genomic target locus, wherein the RNA-guided nuclease creates a double-stranded break in the genomic DNA at the genomic target locus, and the donor polynucleotide generated by the engineered retron is integrated at the genomic target locus recognized by its S′ homology arm and 3′ homology arm by homology directed repair (HDR) HDR with an engineered retron encoding a donor polynucleotide can be used, for example, to create a gene replacement, gene knockout, deletion, insertion, inversion, or point mutation. In some cases, HDR with an engineered retron encoding a donor polynucleotide can be used, for example, to repair a gene, gene knockout, deletion, insertion, inversion, or point mutation. Such methods can thereby create a genetically modified cell. In some embodiments, the method further comprises phenotyping the genetically modified cell or sequencing the genome of the genetically modified cell.

In another aspect, a method of genetically modifying a cell by recombineering is provided, the method comprising: a) transfecting the cell with an engineered retron comprising a heterologous sequence encoding a donor polynucleotide comprising a 5′ homology arm that bybridizes to a 5′ genomic target sequence and a 3′ homology arm that hybridizes to a 3′ genomic target sequence flanking a nucleotide sequence comprising an intended edit to be integrated at a genomic target locus by recombineering; and b) introducing bacteriophage recombination proteins into the cell, wherein the bacteriophage recombination proteins mediate homologous recombination at the target locus such that the donor polynucleotide generated by the engineered retron is integrated at the target locus recognized by its 5′ homology arm and 3′ homology arm to produce a genetically modified cell. Recombineering with an engineered retron encoding a donor polynucleotide can be used, for example, to create a gene replacement, gene knockout, deletion, insertion, inversion, or point mutation. In certain embodiments, the donor polynucleotide is used to modify a plasmid, bacterial artificial chromosome (BAC), or a bacterial chromosome in a bacterial cell by recombineering. In some embodiments, the method further comprises phenotyping the genetically modified cell or sequencing the genome of the genetically modified cell.

In certain embodiments, the bacteriophage recombination proteins are introduced into a bacterial cell by insertion of a replication-defective λ prophage into the bacterial genome. In one embodiment, the bacteriophage comprises exo, bet, and gam genes.

In another aspect, a method of barcoding a cell is provided, the method comprising transfecting a cell with an engineered retron comprising a barcode, as described herein.

In another aspect, a method of producing an in vivo molecular recording system is provided, the method comprising: a) introducing a Cas1 protein or a Cas2 protein of a CRISPR adaptation system into a host cell; b) introducing a CRISPR array nucleic acid sequence comprising a leader sequence and at least one repeat sequence into the host cell, wherein the CRISPR array nucleic acid sequence is integrated into genomic DNA or into a vector in the host cell; and c) introducing a plurality of engineered retrons comprising CRISPR protospacer DNA sequences into the host cell, wherein each retron comprises a different protospacer DNA sequence that can be processed and inserted into the CRISPR array nucleic acid sequence. In certain embodiments, the Cas1 protein or the Cas2 protein are provided by a vector. In certain embodiments, the engineered retron is provided by a vector. In certain embodiments, the plurality of engineered retrons comprises at least three different protospacer DNA sequences.

In another aspect, an engineered cell comprising an in vivo molecular recording system is provided, the engineered cell comprising: a) a Cas1 protein or a Cas2 protein of a CRISPR adaptation system; b) a CRISPR array nucleic acid sequence comprising a leader sequence and at least one repeat sequence into the host cell, wherein the CRISPR array nucleic acid sequence is integrated into genomic DNA or a vector in the engineered cell; and c) a plurality of engineered retrons, each comprising CRISPR protospacer DNA sequences, wherein each retron comprises a different protospacer DNA sequence that can be processed and inserted into the CRISPR array nucleic acid sequence. In certain embodiments, the Cas1 protein or the Cas2 protein are provided by a vector. In certain embodiments, the engineered retron is provided by a vector. In certain embodiments, the plurality of engineered retrons comprises at least three different protospacer DNA sequences.

In another aspect, a kit comprising an engineered cell comprising an in vivo molecular recording system, as described herein, is provided. In some embodiments, the kit further comprises instructions for in vivo molecular recording.

In another aspect, a method of producing recombinant msDNA is provided, the method comprising: a) transfecting a host cell with an engineered retron or vector system described herein; and b) culturing the host cell under suitable conditions, wherein the msDNA is produced.

### BRIEF DESCRIPTION OF THE DRAWINGS

FIGS. 1A-1D show schematics of retron operons and the potential uses of retrons. FIG. 1A shows a schematic of a retron operon that encodes a msr, msd, and reverse transcriptase, where the reverse transcriptase can synthesize a DNA copy of a portion of the msd gene encoding multicopy single-stranded DNA. FIG. 1B illustrates that recombineering is a potential use of retrons, where Beta can protect the ssDNA and can promote annealing of the ssDNA to a complementary ssDNA target, for example, a DNA target in a cell. FIG. 1C illustrates that CRISPR/Cas9 gene editing is a potential use of retrons, where the retron can provide a ssDNA template that can repair a variant or mutant target site. FIG. 1D illustrates that molecular recording is a potential use of retrons (e.g., as provided in WO2018191525 A1, which is specifically incorporated by reference herein in its entirety).

FIGS. 2A-2B shows retron elements and their assembly. FIG. 2A shows retron elements: (1) the msr and the 5′ end of the reverse-transcribed msd are covalently bonded to the priming guanosine via a 2′-5′ linkage, this branched structure impede use in genome engineering, (2) invariant flanking regions that may be required for retron reverse transcription, and so they cannot easily be part of a repair template, (3) a stem that currently is thought to have a limited total length. Another issue for genome engineering is the a native retron poly T stretch that functions as a terminator for Pol III transcription. FIG. 2B illustrates that the non-protein-coding (msr-msd) portion of a retron operon produces a transcript with significant secondary structure, and that the reverse transcriptase (RT) recognizes a particular initiation site in this transcript to then partially reverse transcribe the transcript into RT-DNA (msd).

FIGS. 3A-3D. FIG. 3A shows base structure of wild-type ec86 (also called retron-Ecol ncRNA), after reverse transcription where the msd DNA at the top (SEQ ID NO: 1, GTCAGAAAAAACGGGTTTCCTGGTTGGCTCG GAGAGCATCAGGCGATGCTCTCCGTTCCAACAAGGAAAACAGACAG TAACTCAGA), and the msr RNA is the lower sequence (SEQ ID NO:2-AUGCGCACCCUUAGCGAGAGGUUUAUCAUUAAGGUCAACCUCUG GAUGUUGUUUCGGCAUCCUGCAUUGAAUCUGAGUUACU). FIG. 3B illustrates the quantity of ssDNA produced after expression of ec86, as detected by qPCR analysis of. FIG. 3C shows PAGE analysis of wild-type and variant msd. FIG. 3D shows base structure of two variant msds, the retron-Ecol v32 ncRNA altered from the ec86 wild type (GTCAGAAAAAACGGGTTGTCGCCAGTCTGACTGG CGACAAACAGCTTGTAACTCAGA, SEQ ID NO:3) and retron-Ecol v35 ncRNA that was altered from the v32 ncRNA (GTCAGAAAAAACGGGTGGAGAG GTTGCTGCAACCTCTCCATTTTCTTGTAACTCAGA, SEQ ID NO:4).

FIGS. 4A-4D illustrate an expression system for producing extended msd ssDNAs. FIG. 4A shows an expression construct that splits msr/msd from the retron reverse transcriptase (RT), which permits the production of longer (modified) reverse transcribed msd ssDNA. FIG. 4B shows the arrangement of msr and msd in an expression cassette that is separate from (in trans to) the reverse transcriptase coding region. FIG. 4C illustrates several extensions of the msd ssDNA in the msr/msd expression cassette that is separate from (in trans to) the reverse transcriptase coding region, showing that the msd region can be expanded significantly to include heterologous sequences. FIG. 4D shows PAGE analysis of the msd ssDNA, including the extended msd ssDNA produced as shown in FIGS. 4A-4C.

FIG. 5 shows retron parameters that can be modified.

FIGS. 6A-6F. FIG. 6A schematically illustrates a customized sequencing prep pipeline. The ssDNAs are treated with debranching RNA lariats 1 (DBR1) in the presence of RNase, then a string of polynucleotides of a single type are added using a template independent polymerase (TdT), a complementary strand is generated using an adapter-containing, inverse anchored primers, a second adapter is ligated, and this adapter-linked double-stranded DNA is then indexed and subjected to multiplexed sequencing (SEQ ID NO: 29). FIG. 6B shows that the numbers of nucleotides added by TdT is controllable. FIG. 6C shows an ordered msd ssDNA ec86 v 32 sequence (GTCAGAAAAAACGGGTTGTCGCCAGTCTGACTGGCGACAAACAGCTTGT AACTCAGA, SEQ ID NO:5), illustrating verification by sequencing. FIG. 6D shows a predicted msd ssDNA ec86 v 32 sequence (GTCAGAAAAAACGGGTT GTCGCCAGTCTGACTGGCGACAAACAGCTTGTAACTCAGA, SEQ ID NO:6) illustrating the result of sequencing (GTCAGAAAAAACGGGTTGTCGCCAG TCTGACTGGCGACAAACAGCTTGTAACTCAG, SEQ ID NO:7). FIG. 6E shows a literature wild type msd ssDNA ec86 sequence (GTCAGAAAAAA CGGGTTTCCTGGTTGGCTCGGAGAGCATCAGGCGATGCTCTCTCCGTTCCA ACAAGGAAAACAGACAGTAACTCAGA, SEQ ID NO:8) illustrating the result of sequencing (GTCAGAAAAAACGGGTTTCCTGGTTGGCTCGGAGAGCATCAG GCGATGCTCTCTCCGTTCCAACAAGGAAAACAGACAGTAACTCAG, SEQ ID NO: 9). FIG. 6F shows a literature wild type msd ssDNA ec83 sequence (TTGAAGCCGCGGAACAAACTTTTTGATCCGCAACCTACTGGATTGCGGCT CAAAAAGTTTGTTCCGCAACTGTAAATGTAATC, SEQ ID NO: 10) illustrating the result of sequencing (AGCCGCGGAACAAACTTTTTGATCCGCAACC TACTGGATTGCGGCTCAAAAAGTTTGTTCCGCAACTGTAAATGTAATC, SEQ ID NO:11).

FIGS. 7A-7C-2 illustrate modification of msd DNAs. FIG. 7A schematically illustrates linking of a change in the retron RNA to a barcode that will end up in the msd DNA. FIG. 7B shows increases in ssDNA production from retrons with longer post-msd complementary regions compared to wild type retrons without the longer post-msd regions. FIGS. 7C-1 and 7C-2 illustrate extension and reduction of a region at the 5′ and 3′ ends of a retron non-coding RNA (ncRNA). FIG. 7C-1 schematically illustrates the basic retron structure used, where the complementary region in the ncRNA that is extended is marked with solid black lines while the remaining neRNA is ad dashed line. FIG. 7C-2 graphically illustrates that extension of the neRNA complementary region increases abundance of the RT-DNA relative to a wild-type sequence (where the abundance of the wild-type is 100%), but reduction of the ncRNA complementary region decreases abundance of the RT-DNA. The data shown are from pooled experiments for each variant (n=3, replicates shown).

FIGS. 8A-8B graphically illustrate the quantity of ssDNA can be reduced by shortening of the reverse-transcribed stem of the ncRNA but that extension of the stem does not negatively affect ssDNA production. FIG. 8A schematically illustrates the portion of the neRNA structure modified, which the stem region shown as a solid black line, while the remainder of the neRNA is shown as a dashed line. FIG. 8B graphically illustrates that extension of the neRNA region by about 15-30 nucleotides maintains the abundance of the RT-DNA at about the same levels as observed for the non-extended wild-type ncRNA sequence, however when the length of the ncRNA region is reduced to less than about 14 nucleotides, the amount of ssDNA generated by reverse transcription is reduced compared to the non-extended wild-type ncRNA sequence.

FIGS. 9A-9B illustrate the effects of breaking and fixing the reverse transcribed stem region of the neRNA. FIG. 9A is a schematic diagram of an ncRNA, where the reverse transcribed stem region of the ncRNA is shown as a solid black line. FIG. 9B graphically illustrates the abundance of reversed transcribed DNA of ncRNA structural variants relative to a wild-type sequence. The data are from pooled experiments for each variant. The sequences for the broken stem, fixed stem, and tolerable broken stem ncRNA structural variants are provided in the Examples.

FIGS. 10A-10E illustrate the effects of insertions and deletions in the reverse transcribed region of the ncRNA on the abundance of DNA reverse transcribed from the ncRNA. FIG. 10A schematically illustrates an ncRNA, where the reverse transcribed region of the neRNA is shown as a black dashed and solid line. The dashed line identifies the regions that flank the msd stem. FIG. 10B graphically illustrates the RT-DNA abundance produced by reverse transcription of a series of ncRNA variants, each having a deletion of 3 bases at a distinct position along the msd stem loop, relative to the wild-type sequence. The position of the deletion is plotted along the x-axis. FIG. 10C graphically illustrates the RT-DNA abundance produced by reverse transcription of a series of ncRNA variants, each having an insertion of 3 bases at a distinct position along the msd stem loop, relative to the wild-type sequence. The position of the insertion is plotted along the x-axis. FIG. 10D graphically illustrates the RT-DNA abundance produced by reverse transcription of a series of ncRNA variants, each having a single base change at a distinct position along the msd stem loop, relative to the wild-type sequence. The position of the insertion is plotted along the x-axis. FIG. 10E graphically illustrates the modifiability scores of the msd loop positions in view of the structural changes and results observed for FIGS. 10B-10D. The modifiability scores were based on the average impact of these changes, where the data were from pooled experiments for each variant. Schematics of the stem, loop, and flanking regions are shown in FIGS. 10B-10C for the folded ncRNAs.

FIGS. 11A-11B illustrate use of modified retrons to improve CRISPR-based genomic changes. FIG. 11A is a schematic diagram illustrating integration of retron RT-DNA by the CRISPR integrases Cas1 and Cas2 to modify a genomic CRISPR array. FIG. 11B graphically illustrates that retron-derived spacer DNA can be enhanced by extending the self-complementary region at the 5′ and 3′ ends of the ncRNA.

### DEFINITIONS

The term “about” as used herein when referring to a measurable value such as an amount, a length, and the like, is meant to encompass variations of ±20% or ±10%, more preferably ±5%, even more preferably ±1%, and still more preferably ±0.1% from the specified value.

“Recombinant” as used herein to describe a nucleic acid molecule means a polynucleotide of genomic, cDNA, bacterial, semisynthetic, or synthetic origin which, by virtue of its origin or manipulation, is not associated with all or a portion of the polynucleotide with which it is associated in nature.

The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions. As used herein, a “cell” refers to any type of cell isolated from a prokaryotic, eukaryotic, or archacon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. The methods described herein can be performed, for example, on a sample comprising a single cell or a population of cells. The term also includes genetically modified cells.

The term “transformation” refers to the insertion of an exogenous polynucleotide (e.g., an engineered retron) into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells”, “cells”, “cell lines”, “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences. “Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide or RNA sequence. For example, the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence. The RNA sequence or a portion thereof contains a nucleotide sequence of at least 3 to 5 nucleotides, more preferably at least 8 to 10 nucleotides, and even more preferably at least 15 to 20 nucleotides.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel For a protein that can be subjected to modifications, for example, phosphorylation or glycosylation, different modifications may give rise to different isolated proteins, which can be separately purified.

“Substantially purified” generally refers to isolation of a substance (compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

“Expression” refers to detectable production of a gene product by a cell. The gene product may be a transcription product (i.e., RNA), which may be referred to as “gene expression”, or the gene product may be a translation product of the transcription product (i.e., a protein), depending on the context. “Purified polynucleotide” refers to a polynucleotide of interest or fragment thereof which is essentially free, e.g., contains less than about 50%, preferably less than about 70%, and more preferably less than about at least 90%, of the protein and/or nucleic acids with which the polynucleotide is naturally associated. Techniques for purifying polynucleotides of interest are available in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material and includes uptake of peptide-linked or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Mammalian cell” refers to any cell derived from a mammalian subject suitable for transfection with an engineered retron or vector system comprising an engineered retron, as described herein. The cell may be xenogeneic, autologous, or allogeneic. The cell can be a primary cell obtained directly from a mammalian subject. The cell may also be a cell derived from the culture and expansion of a cell obtained from a mammalian subject Immortalized cells are also included within this definition. In some embodiments, the cell has been genetically engineered to express a recombinant protein and/or nucleic acid.

The term “subject” includes animals, including both vertebrates and invertebrates, including, without limitation, invertebrates such as arthropods, mollusks, annelids, and cnidarians; and vertebrates such as amphibians, including frogs, salamanders, and caecillians; reptiles, including lizards, snakes, turtles, crocodiles, and alligators; fish; mammals, including human and non-human mammals such as non-human primates, including chimpanzees and other apes and monkey species; laboratory animals such as mice, rats, rabbits, hamsters, guinea pigs, and chinchillas; domestic animals such as dogs and cats; farm animals such as sheep, goats, pigs, horses and cows; and birds such as domestic, wild and game birds, including chickens, turkeys and other gallinaceous birds, ducks, geese, and the like. In some cases, the disclosed methods find use in experimental animals, in veterinary application, and in the development of animal models for disease, including, but not limited to, rodents including mice, rats, and hamsters; primates, and transgenic animals.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “barcode” refers to one or more nucleotide sequences that are used to identify a nucleic acid or cell with which the barcode is associated. Barcodes can be 3-1000 or more nucleotides in length, preferably 10-250 nucleotides in length, and more preferably 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. Barcodes may be used, for example, to identify a single cell, subpopulation of cells, colony, or sample from which a nucleic acid originated. Barcodes may also be used to identify the position (i.e., positional barcode) of a cell, colony, or sample from which a nucleic acid originated, such as the position of a colony in a cellular array, the position of a well in a multi-well plate, or the position of a tube, flask, or other container in a rack. For example, a barcode may be used to identify a genetically modified cell from which a nucleic acid originated. In some embodiments, a barcode is used to identify a particular type of genome edit or a particular type of donor nucleic acid.

The terms “hybridize” and “hybridization” refer to the formation of complexes between nucleotide sequences which are sufficiently complementary to form complexes via Watson-Crick base pairing

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequences. Homologous regions may vary in length, but will typically be between 4 and 500 nucleotides (e.g., from about 4 to about 40, from about 40 to about 80, from about 80 to about 120, from about 120 to about 160, from about 160 to about 200, from about 200 to about 240, from about 240 to about 280, from about 280 to about 320, from about 320 to about 360, from about 360 to about 400, from about 400 to about 440, etc.).

As used herein, the terms “complementary” or “complementarity” refers to polynucleotides that are able to form base pairs with one another. Base pairs are typically formed by hydrogen bonds between nucleotide units in an anti-parallel orientation between polynucleotide strands. Complementary polynucleotide strands can base pair in a Watson-Crick manner (e.g., A to T, A to U, C to G), or in any other manner that allows for the formation of duplexes. As persons skilled in the art are aware, when using RNA as opposed to DNA, uracil (U) rather than thymine (T) is the base that is considered to be complementary to adenosine. However, when uracil is denoted in the context of the present invention, the ability to substitute a thymine is implied, unless otherwise stated. “Complementarity” may exist between two RNA strands, two DNA strands, or between an RNA strand and a DNA strand. It is generally understood that two or more polynucleotides may be “complementary” and able to form a duplex despite having less than perfect or less than 100% complementarity. Two sequences are “perfectly complementary” or “100% complementary” if at least a contiguous portion of each polynucleotide sequence, comprising a region of complementarity, perfectly base pairs with the other polynucleotide without any mismatches or interruptions within such region. Two or more sequences are considered “perfectly complementary” or “100% complementary” even if either or both polynucleotides contain additional non-complementary sequences as long as the contiguous region of complementarity within each polynucleotide is able to perfectly hybridize with the other. “Less than perfect” complementarity refers to situations where less than all of the contiguous nucleotides within such region of complementarity are able to base pair with each other. Determining the percentage of complementarity between two polynucleotide sequences is a matter of ordinary skill in the art.

The term “Cas9” as used herein encompasses type II clustered regularly interspaced short palindromic repeats (CRISPR) system Cas9 endonucleases from any species, and also includes biologically active fragments, variants, analogs, and derivatives thereof that retain Cas9 endonuclease activity (i.e., catalyze site-directed cleavage of DNA to generate double-strand breaks). A Cas9 endonuclease binds to and cleaves DNA at a site comprising a sequence complementary to its bound guide RNA (gRNA). For purposes of Cas9 targeting, a gRNA may comprise a sequence “complementary” to a target sequence (e.g., major or minor allele), capable of sufficient base-pairing to form a duplex (i.e., the gRNA hybridizes with the target sequence). Additionally, the gRNA may comprise a sequence complementary to a PAM sequence, wherein the gRNA also hybridizes with the PAM sequence in a target DNA.

The term “donor polynucleotide” refers to a polynucleotide that provides a sequence of an intended edit to be integrated into the genome at a target locus by HDR or recombineering.

A “target site” or “target sequence” is the nucleic acid sequence recognized (i.e., sufficiently complementary for hybridization) by a guide RNA (gRNA) or a homology arm of a donor polynucleotide. The target site may be allele-specific (e.g., a major or minor allele). For example, a target site can be a genomic site that is intended to be modified such as by insertion of one or more nucleotides, replacement of one or more nucleotides, deletion of one or more nucleotides, or a combination thereof.

By “homology arm” is meant a portion of a donor polynucleotide that is responsible for targeting the donor polynucleotide to the genomic sequence to be edited in a cell. The donor polynucleotide typically comprises a 5′ homology arm that bybridizes to a 5′ genomic target sequence and a 3′ homology arm that hybridizes to a 3′ genomic target sequence flanking a nucleotide sequence comprising the intended edit to the genomic DNA. The homology arms are referred to herein as 5′ and 3′ (i.e, upstream and downstream) homology arms, which relates to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively. For example, the nucleotide sequence comprising the intended edit can be integrated into the genomic DNA by HDR or recombineering at the genomic target locus recognized (i.e., sufficiently complementary for hybridization) by the 5′ and 3′ homology arms.

In general, “a CRISPR adaptation system” refers collectively to transcripts and other elements involved in the expression of or directing the activity of CRISPR-associated (“Cas”) genes, including sequences encoding a Cas gene, and a CRISPR array nucleic acid sequence including a leader sequence and at least one repeat sequence. In some embodiments, one or more elements of a CRISPR adaption system are derived from a type I, type II, or type III CRISPR system. Cas1 and Cas2 are found in all three types of CRISPR-Cas systems, and they are involved in spacer acquisition. In the I-E system of E. coli, Cas1 and Cas2 form a complex where a Cas2 dimer bridges two Cas1 dimers. In this complex Cas2 performs a non-enzymatic scaffolding role, binding double-stranded fragments of invading DNA, while Cas1 binds the single-stranded flanks of the DNA and catalyzes their integration into CRISPR arrays. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In some embodiments, a vector comprises a regulatory element operably linked to an enzyme-coding sequence encoding a CRISPR enzyme, such as a Cas protein. Non-limiting examples of Cas proteins include Cas1, CaslB, Cas2, Cas3, Cas4, Cas5, Cas6, Cas7, Cas8, Cas9 (also known as Csnl and Csx12), CasIO, Csyl, Csy2, Csy3, Csel, Cse2, Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, CsxlO, Csx16, CsaX, Csx3, Csxl, Csx15, Csfl, Csf2, Csf3, Csf4, homologs thereof, or modified versions thereof

In certain embodiments, the disclosure provides protospacers that are adjacent to short (3-5 bp) DNA sequences termed protospacer adjacent motifs (PAM). The PAMs are important for type I and type II systems during acquisition. In type I and type II systems, protospacers are excised at positions adjacent to a PAM sequence, with the other end of the spacer is cut using a ruler mechanism, thus maintaining the regularity of the spacer size in the CRISPR array. The conservation of the PAM sequence differs between CRISPR-Cas systems and may be evolutionarily linked to Cas1 and the leader sequence.

In some embodiments, the disclosure provides for integration of defined synthetic DNA that is produced within a cell such as by using an engineered retron system within the cell into a CRISPR array in a directional manner, occurring preferentially, but not exclusively, adjacent to the leader sequence. In the type I-E system from E. coli, it was demonstrated that the first direct repeat, adjacent to the leader sequence is copied, with the newly acquired spacer inserted between the first and second direct repeats.

In one embodiment, the protospacer is a defined synthetic DNA. In some embodiments, the defined synthetic DNA is at least 3, 5,10, 20, 30, 40, or 50nucleotides, or between 3-50, or between 10-100, or between 20-90, or between 30-80, or between 40-70, or between 50-60, nucleotides in length. In one embodiment, the oligo nucleotide sequence or the defined synthetic DNA includes a modified “AAG” protospacer adjacent motif (PAM).

In some embodiments, a regulatory element is operably linked to one or more elements of a CRISPR system so as to drive expression of the one or more elements of the CRISPR system. In general, CRISPRs (Clustered Regularly Interspaced Short

Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al, J. BacterioL, 169:5429-5433(1987); and Nakata et al., J. BacterioL, 171:3553-3556 (1989)), and associated genes. Similar interspersed SSRs have been identified in Haloferax mediterranei, Streptococcus pyogenes, Anabaena, and Mycobacterium tuberculosis (See, Groenen et al., Mol Microbiol., 10:1057-1065 (1993); Hoe et al., Emerg. Infect. Dis., 5:254-263 (1999); Masepohl et al, Biochim. Biophys. Acta 1307:26-30 (1996); and Mojica et al, Mol. Microbiol, 17:85-93 (1995)). The CRISPR loci typically differ from other SSRs by the structure of the repeats, which have been termed short regularly spaced repeats (SRSRs) (Janssen et al, OMICS J. Integ. Biol., 6:23-33 (2002), and Mojica et al, Mol. Microbiol., 36:244-246 (2000)). In general, the repeats are short elements that occur in clusters that are regularly spaced by unique intervening sequences with a substantially constant length (Mojica et al., (2000), supra). Although the repeat sequences are highly conserved between strains, the number of interspersed repeats and the sequences of the spacer regions typically differ from strain to strain (van Embden et al., J. Bacteriol., 182:2393-2401 (2000)). CRISPR loci have been identified in more than 40 prokaryotes (See e.g, Jansen et al, Mol. Microbiol., 43:1565-1575 (2002); and Mojica et al, (2005)) including, but not limited to Aeropyrum, Pyrobaculum, Sulfolobus, Archaeoglobus. Halocarcula, Methanobacteriumn, Methanococcus, Methanosarcina, Methanopyrus, Pyrococcus. Pierophilus, Thernioplasnia, Corynebacterium, Mycobacterium, Streptomyces, Aquifrx, Porphvromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonas, Yersinia, Treponema, and Thermotoga.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about one or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

“Administering” a nucleic acid, such as an engineered retron construct or vector comprising an engineered retron construct to a cell comprises transducing, transfecting, electroporating, translocating, fusing, phagocytosing, shooting or ballistic methods, etc., i.e., any means by which a nucleic acid can be transported across a cell membrane.

Before the present disclosure is further described, it is to be understood that the disclosed subject matter is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosed subject matter. The upper and lower limits of these smaller ranges may independently be included in the smaller ranges, and are also encompassed within the disclosed subject matter, subject to any specifically excluded limit in the stated range. Where the stated range includes one or both of the limits, ranges excluding either or both of those included limits are also included in the disclosed subject matter.

Unless defined otherwise, all technical and scientific terms used herein have the same meaning as commonly understood by one of ordinary skill in the art to which the disclosed subject matter belongs. Although any methods and materials similar or equivalent to those described herein can also be used in the practice or testing of the disclosed subject matter, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a cell” includes a plurality of such cells and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of any features or elements described herein, which includes use of a “negative” limitation.

It is appreciated that certain features of the disclosed subject matter, which are, for clarity, described in the context of separate embodiments, may also be provided in combination in a single embodiment. Conversely, various features of the disclosed subject matter, which are, for brevity, described in the context of a single embodiment, may also be provided separately or in any suitable sub-combination. All combinations of the embodiments pertaining to the disclosure are specifically embraced by the disclosed subject matter and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the disclosed subject matter is not entitled to antedate such publication. Further, the dates of publication provided may be different from the actual publication dates which may need to be independently confirmed.

### DETAILED DESCRIPTION

Engineered retrons, modified to enhance production of multicopy single-stranded DNA (msDNA), are provided. In addition, vector systems encoding such engineered retrons and methods of using engineered retrons and vector systems encoding them in various applications such as CRISPR/Cas-mediated genome editing, recombineering, cellular barcoding, and molecular recording are also provided.

### Engineered Retrons

The present disclosure provides an engineered retron that is modified to enhance production of msDNA in a cell. The engineered retron comprises a pre-msr sequence, an msr gene encoding multicopy single-stranded RNA (msRNA); an msd gene encoding multicopy single-stranded DNA (msDNA); a post-msd sequence and a ret gene encoding a reverse transcriptase. Synthesis of DNA by the retron-encoded reverse transcriptase results can provide a DNA/RNA chimeric product which is composed of single-stranded DNA encoded by the msd gene linked to single-stranded RNA encoded by the msr gene. The retron msr RNA contains a conserved guanosine residue at the end of a stem loop structure. A strand of the msr RNA is joined to the 5′ end of the msd single-stranded DNA by a 2′-5′ phosphodiester linkage at the 2′ position of this conserved guanosine residue.

In the engineered retron, the post-msd sequence is, for example, modified within its self-complementary region (which has sequence complementarity to the pre-msr sequence), wherein the length of the self-complementary region is lengthened relative to the corresponding region of a native retron. Such modifications result in an engineered retron that provides enhanced production of msDNA. In certain embodiments, the complementary region has a length at least 1, at least 2, at least 4, at least 6, at least 8, at least 10, at least 12, at least 14, at least 16, at least 18, at least 20, at least 30, at least 40, or at least 50 nucleotides longer than the wild-type self-complementary region. For example, the self-complementary region may have a length ranging from 1 to 50 nucleotides longer than the native or wild-type complementary region, including any length within this range, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 34, 35, 36, 37, 38, 39, 40, 41, 42, 43, 44, 45, 46, 47,48, 49, or 50 nucleotides longer. In certain embodiments, the self-complementary region has a length ranging from 1 to 16 nucleotides longer than the wild-type complementary region. The single-stranded DNA generated by the engineered retron can be used in various applications.

To create more abundant ssDNA, for example, the ncRNA SEQ ID NO: 12 sequence shown below, with the native self-complementary 3′ and S′ ends highlighted in bold (at positions 1-12 and 158-169), can be extended at positions 1 and 169.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGGAGAGCA TCGCCTGATG |
| 161 | CTCTCCGAGC CAACCAGGAA ACCCGTTTTT TCTGACGTAA |
| 201 | GGGTGCGCA |

For example, as shown below for the following engineered “ncRNA extended” (SEQ ID NO:13), where the additional nucleotides that extend the self-complementary region are shown in italics with underlining.

| | |
|---|---|
| 1 | TGATAAGATT CCGTATGCGC ACCCTTAGCG AGAGGTTTAT |
| 41 | CATTAAGGTC AACCTCTGGA TGTTGTTTCG GCATCCTGCA |
| 81 | TTGAATCTGA GTTACTGTCT GTTTTCCTTG TTGGAACGGA |
| 121 | GAGCATCGCC TGATGCTCTC CGAGCCAACC AGGAAACCCG |
| 161 | TTTTTTCTGA CGTAAGGGTG CGCATACGGA ATCTTATCA |

In some cases, the additional nucleotides can be added to any position in the self-complementary region, for example, anywhere within positions 1-12 and 158-169 of the SEQ ID NO: 12 sequence.

In certain embodiments, sequences of the msr gene, msd gene, and ret gene used in the engineered retron may be derived from a bacterial retron operon. Representative retrons are available such as those from gram-negative bacteria including, without limitation, myxobacteria retrons such as Myxococcus xanthus retrons (e.g., Mx65, Mx162) and Stigmatella aurantiaca retrons (e.g., Sa163); Escherichia coli retrons (e.g., Ec48, E67, Ec73, Ec78, EC83, EC86, EC107, and Ec107); Salmonella enterica, Vibrio cholerae retrons (e.g., Vc81, Vc95, Vc137); Vibrio parahaemolyticus (e.g., Ve96); and Nannocystis exedens retrons (e.g., Ne144). Retron msr gene, msd gene, and ret gene nucleic acid sequences as well as retron reverse transcriptase protein sequences may be derived from any source. Representative retron sequences, including msr gene, msd gene, and ret gene nucleic acid sequences and reverse transcriptase protein sequences are listed in the National Center for Biotechnology Information (NCBI) database. See, for example, NCBI entries: Accession Nos. EF428983, M55249, EU250030, X60206, X62583, AB299445, AB436696, AB436695, M86352, M30609, M24392, AF427793, AQ3354, and AB079134; all of which sequences (as entered by the date of filing of this application) are herein incorporated by reference in their entireties. Any of these retron sequences or a variant thereof comprising a sequence can include variant nucleotides, added nucleotides, or fewer nucleotides. For example, the retrons can have at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity to any of the retron sequences described herein (including those defined by accession number), and can be used to construct an engineered retron or vector system comprising an engineered retron, as described herein.

In some embodiments, recombinant retron constructs have a non-native configuration with a non-native spacing between the msr gene, msd gene, and ret gene. The msr gene and the msd gene may be separated in a trans arrangement rather than provided in the endogenous cis arrangement. In addition, the ret gene may be provided in a trans arrangement with respect to either the msr gene or the msd gene. In some embodiments, the ret gene is provided in a trans arrangement that eliminates a cryptic stop signal for the reverse transcriptase, which allows the generation of longer single stranded DNAs from the engineered retron construct.

In some embodiments, the retron construct is modified with respect to the native retron to include a heterologous sequence of interest. In this context, the retrons can be engineered with heterologous sequences for use in a variety of applications. For example, heterologous sequences can be added to retron constructs to provide a cell with a nucleic acid encoding a protein or regulatory RNA of interest, a donor polynucleotide suitable for use in gene editing, e.g., by homology directed repair

(HDR) or recombination-mediated genetic engineering (recombineering), or a CRISPR protospacer DNA sequence for use in molecular recording, as discussed further below. Such heterologous sequences may be inserted, for example, into the msr gene or the msd gene such that the heterologous sequence is transcribed by the retron reverse transcriptase as part of the msDNA product.

In some cases, the heterologous sequence of interest can be inserted into the loop of the msd stem loop.

For example, the engineered retrons can include a unique barcode to facilitate multiplexing. Barcodes may comprise one or more nucleotide sequences that are used to identify a nucleic acid or cell with which the barcode is associated. Such barcodes may be inserted for example, into the loop region of the msd-encoded DNA. Barcodes can be 3-1000 or more nucleotides in length, preferably 10-250 nucleotides in length, and more preferably 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length. In some embodiments, barcodes are also used to identify the position (i.e., positional barcode) of a cell, colony, or sample from which a retron originated, such as the position of a colony in a cellular array, the position of a well in a multi-well plate, the position of a tube in a rack, or the location of a sample in a laboratory. In particular, a barcode may be used to identify the position of a genetically modified cell containing a retron. The use of barcodes allows retrons from different cells to be pooled in a single reaction mixture for sequencing while still being able to trace a particular retron back to the colony from which it originated.

In addition, adapter sequences can be added to retron constructs to facilitate high-throughput amplification or sequencing. For example, a pair of adapter sequences can be added at the S′ and 3′ ends of a retron construct to allow amplification or sequencing of multiple retron constructs simultaneously by the same set of primers. Amplification of retron constructs may be performed, for example, before transfection of cells or ligation into vectors. Any method for amplifying the retron constructs may be used, including, but not limited to polymerase chain reaction (PCR), isothermal amplification, nucleic acid sequence-based amplification (NASBA), transcription mediated amplification (TMA), strand displacement amplification (SDA), and ligase chain reaction (LCR). In one embodiment, the retron constructs comprise common 5′ and 3′ priming sites to allow amplification of retron sequences in parallel with a set of universal primers. In another embodiment, a set of selective primers is used to selectively amplify a subset of retron sequences from a pooled mixture.

Delivery of an engineered retron to a cell will generally be accomplished with or without vectors. The engineered retrons (or vectors containing them) may be introduced into any type of cell, including any cell from a prokaryotic, eukaryotic, or archacon organism, including bacteria, archaea, fungi, protists, plants (e.g., monocotyledonous and dicotyledonous plants); and animals (e.g., vertebrates and invertebrates). Examples of animals that may be transfected with an engineered retron include, without limitation, vertebrates such as fish, birds, mammals (e.g., human and non-human primates, farm animals, pets, and laboratory animals), reptiles, and amphibians. Examples of plants that may be transfected with an engineered retron include, without limitation, crops including cereals such as wheat, oats, and rice, legumes such as soybeans and peas, corn, grasses such as alfalfa, and cotton. The engineered retrons can be introduced into a single cell or a population of cells of interest. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be transfected with the engineered retrons. The subject methods are also applicable to cellular fragments, cell components, or organelles (e.g., mitochondria in animal and plant cells, plastids (e.g., chloroplasts) in plant cells and algae). Cells may be cultured or expanded after transfection with the engineered retron constructs.

Methods of introducing nucleic acids into a host cell are well known in the art. Commonly used methods include chemically induced transformation, typically using divalent cations (e.g., CaCl₂), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising engineered retrons into nuclei. See, e.g., Sambrook et al. (2001) Molecular Cloning, a laboratory manual, 3^{rd} edition, Cold Spring Harbor Laboratories, New York, Davis et al. (1995) Basic Methods in Molecular Biology, 2^{nd} edition, McGraw-Hill, and Chu et al. (1981) Gene 13:197; herein incorporated by reference in their entireties.

### Vector Sestems Comprising Engineered Retrons

In certain embodiments, the retron msr gene, msd gene, and ret gene are expressed in vivo from a vector within a cell. A “vector” is a composition of matter which can be used to deliver a nucleic acid of interest to the interior of a cell. The retron msr gene, msd gene, and ref gene can be introduced into a cell with a single vector or in multiple separate vectors to produce msDNA in a host subject. Vectors typically include control elements operably linked to the retron sequences, which allow for the production of msDNA in vivo in the subject species. For example, the retron msr gene, msd gene, and ret gene can be operably linked to a promoter to allow expression of the retron reverse transcriptase and msDNA product. In some embodiments, heterologous sequences encoding desired products of interest (e.g., polynucleotide encoding polypeptide or regulatory RNA, donor polynucleotide for gene editing, or protospacer DNA for molecular recording) may be inserted in the msr gene or msd gene. Any eukaryotic, archeon, or prokaryotic cell, capable of being transfected with a vector comprising the engineered retron sequences, may be used to produce the msDNA. The ability of constructs to produce the msDNA along with other retron-encoded products can be empirically determined.

In some embodiments, the engineered retron is produced by a vector system comprising one or more vectors. In the vector system, the msr gene, the msd gene, and the ret gene may be provided by the same vector (i.e., cis arrangement of all such retron elements), wherein the vector comprises a promoter operably linked to the msr gene and the msd gene. In some embodiments, the promoter is further operably linked to the ret gene In other embodiments, the vector further comprises a second promoter operably linked to the ret gene. Alternatively, the ret gene may be provided by a second vector that does not include the msr gene and the msd gene (i.e., trans arrangement of msr-msd and ret). In yet other embodiments, the msr gene, the msd gene, and the ret gene are each provided by different vectors (i.e., trans arrangement of all retron elements). Numerous vectors are available including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In certain embodiments, the nucleic acid comprising an engineered retron sequence is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al.,Cell (1985) 41:521, such as elements included in the CMV intron A sequence.

In one embodiment, an expression vector for expressing an engineered retron, including the msr gene, nsd gene, and ret gene comprises a promoter “operably linked” to a polynucleotide encoding the msr gene, msd gene, and ret gene. The phrase “operably linked” or “under transcriptional control” as used herein means that the promoter is in the correct location and orientation in relation to a polynucleotide to control the initiation of transcription by RNA polymerase and expression of the msr gene, msd gene, and ret gene.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences may include UTRs comprising an internal ribosome entry site (IRES).

Inclusion of an IRES permits the translation of one or more open reading frames from a vector. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298, Rees et al., BioTechniques (1996) 20:102-110, Kobayashi et al., BioTechniques (1996) 21:399-402; and Mosser et al., BioTechniques (1997 22 150-161.A multitude of IRES sequences are known and include sequences derived from a wide variety of viruses, such as from leader sequences of picornaviruses such as the encephalomyocarditis virus (EMCV) UTR (Jang et al. J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100(25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279 (5): 3389-3397), and the like A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17): 7622-7635), vascular endothelial growth factor IRES (Baranick et al. (2008) Proc. Natl. Acad. Sci. U.S.A. 105(12): 4733-4738, Stein et al. (1998) Mol. Cell. Biol. 18(6): 3112-3119, Bert et al. (2006) RNA 12(6): 1074-1083), and insulin-like growth factor 2 IRES (Pedersen et al. (2002) Biochem. J. 363 (Pt 1): 37-44). These elements are readily commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express multiple bacteriophage recombination proteins for recombineering or an RNA-guided nuclease (e.g., Cas9) for HDR in combination with a retron reverse transcriptase from an expression cassette

Alternatively, a polynucleotide encoding a viral T2A peptide can be used to allow production of multiple protein products (e.g., Cas9, bacteriophage recombination proteins, retron reverse transcriptase) from a single vector. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Thosea asigna virus and porcine teschovirus-1. See, e.g., Kim et al. (2011) PLOS One 6 (4): e18556, Trichas et al. (2008) BMC Biol. 6:40, Provost et al. (2007) Genesis 45 (10): 625-629, Furler et al. (2001) Gene Ther. 8 (11): 864-873; herein incorporated by reference in their entireties.

In certain embodiments, the expression construct comprises a plasmid suitable for transforming a bacterial host. Numerous bacterial expression vectors are known to those of skill in the art, and the selection of an appropriate vector is a matter of choice. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31 Bacterial plasmids may contain antibiotic selection markers (e.g., ampicillin, kanamycin, erythromycin, carbenicillin, streptomycin, or tetracycline resistance), a lacZ gene (β-galactosidase produces blue pigment from x-gal substrate), fluorescent markers (e.g., GFP. mCherry), or other markers for selection of transformed bacteria. See, c.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, MET15, ura4+, leu1+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E. coli) and yeast cells. A number of different types of yeast plasmids are available including yeast integrating plasmids (YIp), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct comprises a virus or engineered construct derived from a viral genome. A number of viral based systems have been developed for gene transfer into mammalian cells. These include adenoviruses, retroviruses (y-retroviruses and lentiviruses), poxviruses, adeno-associated viruses, baculoviruses, and herpes simplex viruses (see e.g., Warnock et al. (2011) Methods Mol. Biol. 737:1-25; Walther et al. (2000) Drugs 60(2): 249-271; and Lundstrom (2003) Trends Biotechnol. 21(3): 117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24): 2516-2527) Lentiviruses are a class of retroviruses that are particularly useful for delivering polynucleotides to mammalian cells because they are able to infect both dividing and nondividing cells (see e.g., Lois et al (2002) Science 295:868-872; Durand et al. (2011) Viruses 3(2): 132-159; herein incorporated by reference).

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the engineered retrons is the enterically administered recombinant poxvirus vaccines described by Small, Jr., P. A., et al. (U.S. Pat. No. 5,676,950, issued Oct. 14, 1997, herein incorporated by reference).

Additional viral vectors which will find use for delivering the nucleic acid molecules of interest include those derived from the pox family of viruses, including vaccinia virus and avian poxvirus. By way of example, vaccinia virus recombinants expressing a nucleic acid molecule of interest (e.g., engineered retron) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

Alternatively, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268.6866-6869 and Wagner et al., Proc. Natl. Acad. Sci. USA (1992) 89:6099-6103, can also be used for gene delivery. Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843, 723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77:10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered retron) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a cDNA whose expression is under the control of the T7 promoter. Thus, some of the T7 RNA polymerase generated from translation of the amplification template RNA will lead to transcription of the desired gene Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2^{nd} edition, 2007) and L. King The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant cells. Generally, such systems use virus-based vectors to transfect plant cells with heterologous genes. For a description of such systems see, e.g., Porta et al., Mol. Biotech. (1996) 5:209-221; and Hackland et al.,Arch. Virol. (1994) 139:1-22.

In order to effect expression of engineered retron constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

Several non-viral methods for the transfer of expression constructs into cultured cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell

Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad Sci. USA 81:7161-7165), Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352;

Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Once the expression construct has been delivered into the cell the nucleic acid comprising the engineered retron sequence may be positioned and expressed at different sites. In certain embodiments, the nucleic acid comprising the engineered retron sequence may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids comprising the engineered retron. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding an engineered retron of interest may also be transferred in a similar manner in vivo and express retron products.

In still another embodiment, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In certain embodiments, the liposome may be complexed with a hemagglutinating virus (HVJ) This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6): 3361-3364). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9): 3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91 (9): 4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosyl-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In a particular example, a recombinant polynucleotide comprising an engineered retron may be administered in combination with a cationic lipid. Examples of cationic lipids include, but are not limited to, lipofectin, DOTMA, DOPE, and DOTAP. The publication of WO/0071096, which is specifically incorporated by reference, describes different formulations, such as a DOTAP: cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In certain embodiments, gene transfer may more easily be performed under ex vivo conditions. Ex vivo gene therapy refers to the isolation of cells from a subject, the delivery of a nucleic acid into cells in vitro, and then the return of the modified cells back into the subject. This may involve the collection of a biological sample comprising cells from the subject. For example, blood can be obtained by venipuncture, and solid tissue samples can be obtained by surgical techniques according to methods well known in the art

Usually, but not always, the subject who receives the cells (i.e., the recipient) is also the subject from whom the cells are harvested or obtained, which provides the advantage that the donated cells are autologous. However, cells can be obtained from another subject (i.e., donor), a culture of cells from a donor, or from established cell culture lines. Cells may be obtained from the same or a different species than the subject to be treated, but preferably are of the same species, and more preferably of the same immunological profile as the subject. Such cells can be obtained, for example, from a biological sample comprising cells from a close relative or matched donor, then transfected with nucleic acids (e g., comprising an engineered retron), and administered to a subject in need of genome modification, for example, for treatment of a disease or condition.

### Kits

Also provided are kits comprising engineered retron constructs as described herein. In some embodiments, the kit provides an engineered retron construct or a vector system comprising such a retron construct. In some embodiments, the engineered retron construct, included in the kit, comprises a heterologous sequence capable of providing a cell with a nucleic acid encoding a protein or regulatory RNA of interest, a cellular barcode, a donor polynucleotide suitable for use in gene editing, e.g., by homology directed repair (HDR) or recombination-mediated genetic engineering (recombineering), or a CRISPR protospacer DNA sequence for use in molecular recording. Other agents may also be included in the kit such as transfection agents, host cells, suitable media for culturing cells, buffers, and the like. In the context of a kit, agents can be provided in liquid or sold form in any convenient packaging (e.g., stick pack, dose pack, etc.). The agents of a kit can be present in the same or separate containers. The agents may also be present in the same container. In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

Retrons can be engineered with heterologous sequences for use in a variety of applications. For example, heterologous sequences can be added to retron constructs to provide a cell with a heterologous nucleic acid encoding a protein or regulatory RNA of interest, a cellular barcode, a donor polynucleotide suitable for use in gene editing, e.g., by homology directed repair (HDR) or recombination-mediated genetic engineering (recombineering), or a CRISPR protospacer DNA sequence for use in molecular recording, as discussed further below. Such heterologous sequences may be inserted, for example, into the msr gene or the msd gene such that the heterologous sequence is transcribed by the retron reverse transcriptase as part of the msDNA product.

### Production of Protein or RNA

For example, the single-stranded DNA generated by an engineered retron can be used to produce a desired product of interest in cells. In some embodiments, the retron is engineered with a heterologous sequence encoding a polypeptide of interest to allow production of the polypeptide from the retron msDNA generated in a cell The polypeptide of interest may be any type of protein/peptide including, without limitation, an enzyme, an extracellular matrix protein, a receptor, transporter, ion channel, or other membrane protein, a hormone, a neuropeptide, an antibody, or a cytoskeletal protein; or a fragment thereof, or a biologically active domain of interest. In some embodiments, the protein is a therapeutic protein or therapeutic antibody for use in treatment of a disease.

In other embodiments, the retron is engineered with a heterologous sequence encoding an RNA of interest to allow production of the RNA from the retron in a cell. The RNA of interest may be any type of RNA including, without limitation, a RNA interference (RNAi) nucleic acid or regulatory RNA such as, but not limited to, a microRNA (miRNA), a small interfering RNA (siRNA), a short hairpin RNA (shRNA), a small nuclear RNA (snRNA), a long non-coding RNA (IncRNA), an antisense nucleic acid, and the like.

### Gene Editing

In some embodiments, the retron is engineered with a heterologous sequence encoding a donor polynucleotide suitable for use with a CRISPR/Cas genome editing system. Donor polynucleotides comprise a sequence comprising an intended genome edit flanked by a pair of homology arms responsible for targeting the donor polynucleotide to the target locus to be edited in a cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relate to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit can be integrated into the genomic DNA by HDR at the genomic target locus recognized (i.e., having sufficient complementary for hybridization) by the 5′ and 3′ homology arms.

In certain embodiments, the corresponding homologous nucleotide sequences in the genomic target sequence (i.c., the “5′ target sequence” and “3′ target sequence”) flank a specific site for cleavage and/or a specific site for introducing the intended edit. The distance between the specific cleavage site and the homologous nucleotide sequences (e.g, each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In a preferred embodiment, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g. 10 nucleotides or more, 15nucleotides or more, 20 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000nucleotides (1 kb) or more, 5000 nucleotides (5 kb) or more, 10000 nucleotides (10 kb) or more, etc. In some instances, the 5′ and 3′ homology arms are substantially equal in length to one another. However, in some instances the 5′ and 3′ homology arms are not necessarily equal in length to one another. For example, one homology arm may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g. one may be 40% shorter or more, 50% shorter or more, sometimes 60% shorter or more, 70% shorter or more, 80% shorter or more, 90% shorter or more, or 95% shorter or more than the other homology arm.

The donor polynucleotide is used in combination with an RNA-guided nuclease, which is targeted to a particular genomic sequence (i.e., genomic target sequence to be modified) by a guide RNA. A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to the sequence of a minor allele to target the nuclease-gRNA complex to the site of a mutation. The mutation may comprise an insertion, a deletion, or a substitution. For example, the mutation may include a single nucleotide variation, gene fusion, translocation, inversion, duplication, frameshift, missense, nonsense, or other mutation associated with a phenotype or disease of interest. The targeted minor allele may be a common genetic variant or a rare genetic variant. In certain embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to remove the mutation from a gene. Alternatively, the gRNA can be designed with a sequence complementary to the sequence of a major or wild-type allele to target the nuclease-gRNA complex to the allele for the purpose of genome editing to introduces a mutation into a gene in the genomic DNA of the cell, such as an insertion, deletion, or substitution Such genetically modified cells can be used, for example, to alter phenotype, confer new properties, or produce disease models for drug screening.

In certain embodiments, the RNA-guided nuclease used for genome modification is a clustered regularly interspersed short palindromic repeats (CRISPR) system Cas nuclease. Any RNA-guided Cas nuclease capable of catalyzing site-directed cleavage of DNA to allow integration of donor polynucleotides by the HDR mechanism can be used in genome editing, including CRISPR system type I, type II, or type III Cas nucleases. Examples of Cas proteins include Cas1, Cas1B, Cas2, Cas3, Cas4, Cas5, Casse (CasD), Cas6, Cas6e, Casof, Cas7, Cas8al, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, a type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP_002989955, WP 038434062, WP_011528583); Campylobacter jejuni (WP_022552435, YP_002344900), Campylobacter coli (WP_060786116); Campylobacter fetus (WP_059434633); Corynebacterium ulcerans (NC_015683, NC_017317); Corynebacterium diphtheria (NC_016782, NC_016786); Enterococcus faecalis (WP_033919308); Spiroplasma syrphidicola (NC_021284); Prevotella intermedia (NC_017861); Spiroplasma taiwanense (NC_021846); Streptococcus iniae (NC_021314); Belliella baltica (NC_018010); Psychroflexus torquis (NC_018721); Streptococcus thermophilus (YP_820832), Streptococcus mutans (WP_ 061046374, WP_024786433); Listeria innocua (NP_472073); Listeria monocytogenes (WP_061665472); Legionella pneumophila (WP_062726656); Staphylococcus aureus (WP_001573634); Francisella tularensis (WP_032729892, WP_014548420), Enterococcus faecalis (WP_033919308); Lactobacillus rhamnosus (WP_048482595, WP_032965177); and Neisseria meningitidis (WP_061704949, YP_002342100); all of which sequences (as entered by the date of filing of this application) are herein incorporated by reference in their entireties. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42 (4): 2577-90; Kapitonov et al. (2015) J. Bacteriol. 198(5): 797-807, Shmakov et al. (2015) Mol. Cell. 60(3): 385-397, and Chylinski et al. (2014) Nucleic Acids Res. 42 (10): 6091-6105); for sequence comparisons and a discussion of genetic diversity and phylogenetic analysis of Cas9.

The CRISPR-Cas system naturally occurs in bacteria and archaea where it plays a role in RNA-mediated adaptive immunity against foreign DNA. The bacterial type II CRISPR system uses the endonuclease, Cas9, which forms a complex with a guide RNA (gRNA) that specifically hybridizes to a complementary genomic target sequence, where the Cas9 endonuclease catalyzes cleavage to produce a double-stranded break. Targeting of Cas9 typically further relies on the presence of a S′ protospacer-adjacent motif (PAM) in the DNA at or near the gRNA-binding site.

The genomic target site will typically comprise a nucleotide sequence that is complementary to the gRNA and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide. In certain embodiments, the allele targeted by a gRNA comprises a mutation that creates a PAM within the allele, wherein the PAM promotes binding of the Cas9-gRNA complex to the allele.

In certain embodiments, the gRNA is 5-50 nucleotides, 10-30 nucleotides, 15-25nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1) is used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5nucleotide overhang. For a discussion of Cpf1, see, e.g., Ledford et al. (2015) Nature. 526 (7571): 17-17, Zetsche et al. (2015) Cell. 163 (3): 759-771, Murovec et al. (2017) Plant Biotechnol. J. 15(8): 917-926, Zhang et al. (2017) Front. Plant Sci. 8:177, Fernandes et al. (2016) Postepy Biochem. 62(3): 315-326; herein incorporated by reference.

C2c1 is another class II CRISPR/Cas system RNA-guided nuclease that may be used. C2c1, similarly to Cas9, depends on both a crRNA and tracrRNA for guidance to target sites. For a description of C2c1, see, e.g., Shmakov et al. (2015) Mol Cell. 60 (3): 385-397, Zhang et al. (2017) Front Plant Sci. 8:177; herein incorporated by reference.

In yet another embodiment, an engineered RNA-guided FokI nuclease may be used. RNA-guided FokI nucleases comprise fusions of inactive Cas9 (dCas9) and the FokI endonuclease (FokI-dCas9), wherein the dCas9 portion confers guide RNA-dependent targeting on FokI. For a description of engineered RNA-guided FokI nucleases, see, e.g., Havlicek et al. (2017) Mol. Ther. 25(2): 342-355, Pan et al. (2016) Sci Rep. 6:35794, Tsai et al. (2014) Nat Biotechnol. 32(6): 569-576; herein incorporated by reference.

The RNA-guided nuclease can be provided in the form of a protein, optionally where the nuclease complexed with a gRNA, or provided by a nucleic acid encoding the RNA-guided nuclease, such as an RNA (e.g., messenger RNA) or DNA (expression vector) In some embodiments, the RNA-guided nuclease and the gRNA are both provided by vectors. Both can be expressed by a single vector or separately on different vectors. The vector(s) encoding the RNA-guided nuclease an gRNA may be included in the vector system comprising the engineered retron msr gene, msd gene and ret gene sequences.

Codon usage may be optimized to improve production of an RNA-guided nuclease and/or retron reverse transcriptase in a particular cell or organism. For example, a nucleic acid encoding an RNA-guided nuclease or reverse transcriptase can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease or reverse transcriptase is introduced into cells, the protein can be transiently, conditionally, or constitutively expressed in the cell.

### Recombineering

Recombineering (recombination-mediated genetic engineering) can be used in modifying chromosomal as well as episomal replicons in cells, for example, to create gene replacements, gene knockouts, deletions, insertions, inversions, or point mutations. Recombineering can also be used to modify a plasmid or bacterial artificial chromosome (BAC), for example, to clone a gene or insert markers or tags. The engineered retrons described herein can be used in recombineering applications to provide linear single-stranded or double-stranded DNA for recombination. Homologous recombination is mediated by bacteriophage proteins such as RecE/RecT from Rac prophage or Redαβδ from bacteriophage lambda. The linear DNA should have sufficient homology at the 5′ and 3′ ends to a target DNA molecule present in a cell (e.g., plasmid, BAC, or chromosome) to allow recombination.

The linear double-stranded or single-stranded DNA molecule used in recombineering (i.e. donor polynucleotide) comprises a sequence having the intended edit to be inserted flanked by two homology arms that target the linear DNA molecule to a target site for homologous recombination. Homology arms for recombineering typically range in length from 13-300 nucleotides, or 20 to 200 nucleotides, including any length within this range such as 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 nucleotides in length. In some embodiments, a homology arm is at least 15, at least 20, at least 30, at least 40, or at least 50 or more nucleotides in length. Homology arms ranging from 40-50 nucleotides in length generally have sufficient targeting efficiency for recombination; however, longer homology arms ranging from 150 to 200 bases or more may further improve targeting efficiency. In some embodiments, the S′ homology arm and the 3′ homology arm differ in length. For example, the linear DNA may have about 50 bases at the 5′ end and about 20 bases at the 3′ end with homology to the region to be targeted.

The bacteriophage homologous recombination proteins can be provided to a cell as proteins or by one or more vectors encoding the recombination proteins. In some embodiments, one or more vectors encoding the bacteriophage recombination proteins are included in the vector system comprising the engineered retron msr gene, msd gene and ret gene sequences.

Additionally, a number of bacterial strains containing prophage recombination systems are available for recombineering, including, without limitation, DY380, containing a defective A prophage with recombination proteins exo, bet, and gam; EL250, derived from DY380, which in addition to the recombination genes found in DY380, also contains a tightly controlled arabinose-inducible flpe gene (flpe mediates recombination between two identical frt sites); EL350, also derived from DY380, which in addition to the recombination genes found in DY380, also contains a tightly controlled arabinose-inducible cre gene (cre mediates recombination between two identical loxP sites; SW102, derived from DY380, which is designed for BAC recombineering using a galK positive/negative selection; SW105, derived from EL250, which can also be used for galK positive/negative selection, but like EL250, contain an ara-inducible Flpe gene; and SW106, derived from EL350, which can be used for galK positive/negative selection, but like EL350, contains an ara-inducible Cre gene. Recombincering can be carried out by transfecting bacterial cells of such strains with an engineered retron comprising a heterologous sequence encoding a linear DNA suitable for recombineering. For a discussion of recombineering systems and protocols, see, e.g., Sharan et al. (2009) Nat Protoc. 4 (2): 206-223, Zhang et al. (1998) Nature Genetics 20:123-128, Muyrers et al. (1999) Nucleic Acids Res. 27:1555-1557, Yu et al. (2000) Proc. Natl. Acad Sci U.S.A. 97 (11): 5978-5983; herein incorporated by reference.

### Molecular Recording

In some embodiments, the heterologous sequence in the engineered retron construct comprises a synthetic CRISPR protospacer DNA sequence to allow molecular recording. The endogenous CRISPR Cas1-Cas2 system is normally utilized by bacteria and archaea to keep track of foreign DNA sequences originating from viral infections by storing short sequences (i.e., protospacers) that confer sequence-specific resistance to invading viral nucleic acids within genome-based arrays. These arrays not only preserve the spacer sequences but also record the order in which the sequences are acquired, generating a temporal record of acquisition events

This system can be adapted to record arbitrary DNA sequences into a genomic CRISPR array in the form of “synthetic protospacers” that are introduced into cells using engineered retrons. Engineered retrons carrying the protospacer sequences can be used for integration of synthetic CRISPR protospacer sequences at a specific genomic locus by utilizing the CRISPR system Cas1-Cas2 complex. Molecular recording can be used to keep track of certain biological events by producing a stable genetic memory tracking code. See, e.g., Shipman et al. (2016) Science 353 (6298): aaf1175 and International Patent Application Publication No. WO/2018/191525; herein incorporated by reference in their entireties.

In some embodiments, the CRISPR-Cas system is harnessed to record specific and arbitrary DNA sequences into a bacterial genome. The DNA sequences can be produced by an engineered retron within the cell. For example, the engineered retron can be used to produce the protospacers within the cell, which are inserted into a CRISPR array within the cell. The cell may be modified to include one or more engineered retrons (or vector systems encoding them) that can produce one or more synthetic protospacers in the cell, wherein the synthetic protospacers are added to the CRISPR array. A record of defined sequences, recorded over many days, and in multiple modalities can be generated.

In some embodiments, the engineered retron comprises an msd protospacer nucleic acid region or an msr protospacer nucleic acid region In the case of a msr protospacer nucleic acid region, the protospacer sequence is first incorporated into the msr RNA, which is reverse transcribed into protospacer DNA. Double stranded protospacer DNA is produced when two complementary protospacer DNA sequences having complementary sequences hybridize, or when a double-stranded structure (such as a hairpin) is formed in a single stranded protospacer DNA (e.g., a single msDNA can form an appropriate hairpin structure to provide the double stranded DNA protospacer).

In some embodiments, a single stranded DNA produced in vivo from a first engineered retron may be hybridized with a complementary single-stranded DNA produced in vivo from the same retron or a second engineered retron or may form a hairpin structure and then used as a protospacer sequence to be inserted into a CRISPR array as a spacer sequence. The engineered retron(s) should provide sufficient levels of the protospacer sequence within a cell for incorporation into the CRISPR array. The use of protospacers generated within the cell extends the in vivo molecular recording system from only capturing information known to a user, to capturing biological or environmental information that may be previously unknown to a user. For example, an msDNA protospacer sequence in an engineered retron construct may be driven by a promoter that is downstream of a sensor pathway for a biological phenomenon or environmental toxin. The capture and storage of the protospacer sequence in the CRISPR array records the event. If multiple msDNA protospacers are driven by different promoters, the activity of those promoters is recorded (along with anything that may be upstream of the promoters) as well as the relative order of promoter activity (based on the relative position of spacer sequences in the CRISPR array). At any point after the recording has taken place, the CRISPR array may be sequenced to determine whether a given biological or environmental event has taken place and the order of multiple events, given by the presence and relative position of msDNA-derived spacers in the CRISPR array.

In some embodiments, the synthetic protospacer further comprises an AAG PAM sequence at its 5′ end. Protospacers including the S′ AAG PAM are acquired by the CRISPR array with greater efficiency than those that do not include a PAM sequence.

In some embodiments, Cas1 and Cas2 are provided by a vector that expresses the Cas1 and Cas2 at a level sufficient to allow the synthetic protospacer sequences produced by engineered retrons to be acquired by a CRISPR array in a cell. Such a vector system can be used to allow molecular recording in a cell that lacks endogenous Cas proteins.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-60 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. An engineered retron comprising:
   a) a pre-msr sequence;
   b) an msr gene encoding multicopy single-stranded RNA (msRNA);
   c) an msd gene encoding multicopy single-stranded DNA (msDNA);
   d) a post-msd sequence comprising a self-complementary region having sequence complementarity to the pre-msr sequence, wherein the self-complementary region has a length at least 1 to 50 nucleotides longer than a wild-type self-complementary region such that the engineered retron is capable of enhanced production of the msDNA; and
   e) a ret gene encoding a reverse transcriptase.
2. The engineered retron, wherein the self-complementary region has a length at least 5, at least 10, at least 15, at least 20, at least 25, or at least 30 nucleotides longer than the wild-type self-complementary region.
3. The engineered retron of aspect 1 or 2, wherein the msr gene and the msd gene are provided in a trans arrangement or a cis arrangement.
4. The engineered retron of aspect 3, wherein the ret gene is provided in a trans arrangement with respect to the msr gene and the msd gene.
5. The engineered retron of any of aspects 1-4, wherein the msr gene, msd gene, and ret gene are derived from a myxobacteria retron, an Escherichia coli retron, or a Vibrio cholerae retron.
6. The engineered retron of aspect 5, wherein the Escherichia coli retron is EC83 or EC86.
7. The engineered retron of any of aspects 1-6, further comprising a heterologous sequence of interest.
8. The engineered retron of aspect 7, wherein the heterologous sequence is inserted into the msr gene or the msd gene.
9. The engineered retron of aspect 1-7 or 8, wherein a heterologous nucleic acid segment is inserted 9 to 20 bases from the base of the complementary region.
10. The engineered retron of aspect 7, 8 or 9, wherein the heterologous sequence is within the hairpin loop of the msd, post-msd, or msDNA.
11. The engineered retron of aspect 1-8 or 9, wherein a heterologous nucleic acid segment is inserted at position 20 to 60 of the msd or the post-msd.
12. The engineered retron of aspect 7-10 or 11, wherein the heterologous sequence encodes a donor polynucleotide comprising a 5′ homology arm that hybridizes to a 5′ target sequence and a 3′ homology arm that hybridizes to a 3′ target sequence flanking a nucleotide sequence comprising an intended edit to be integrated at a target locus by homology directed repair (HDR) or recombineering.
13. The engineered retron of aspect 7-11 or 12, wherein the heterologous sequence comprises a CRISPR protospacer DNA sequence.
14. The engineered retron of aspect 13, wherein the CRISPR protospacer DNA sequence comprises a modified AAG protospacer adjacent motif (PAM).
15. The engineered retron of any of aspects 1-14, further comprising a barcode sequence.
16. The engineered retron of aspect 15, wherein the barcode sequence is located in a hairpin loop of the msDNA.
17. A vector system comprising one or more vectors comprising the engineered retron of any of aspects 1-16.
18. The vector system of aspect 17, wherein the msr gene and the msd gene are provided by the same vector or different vectors.
19. The vector system of aspect 17 or 18, wherein the msr gene, the msd gene, and the ret gene are provided by the same vector.
20 The vector system of aspect 17, 18 or 19, wherein the vector comprises a promoter operably linked to the msr gene and the msd gene.
21. The vector system of aspect 20, wherein the promoter is further operably linked to the ret gene.
22. The vector system of aspect 20 or 21, further comprising a second promoter operably linked to the ret gene.
23. The vector system of aspect 17-21 or 22, wherein the msr gene, the msd gene, and the ret gene are provided by different vectors.
24. The vector system of any of aspects 17-22 or 23, wherein the one or more vectors are viral vectors or nonviral vectors.
25. The vector system of aspect 24, wherein the nonviral vectors are plasmids.
26. The vector system of any of aspects 17-25, wherein the engineered retron comprises a donor polynucleotide comprising a 5′ homology arm that hybridizes to a S′ target sequence and a 3′ homology arm that hybridizes to a 3′ target sequence flanking a nucleotide sequence comprising an intended edit to be integrated at a target locus by homology directed repair (HDR) or recombineering.
27. The vector system of aspect 26, further comprising a vector encoding an RNA-guided nuclease.
28. The vector system of aspect 27, wherein the RNA-guided nuclease is a Cas nuclease or an engineered RNA-guided Fokl-nuclease.
29. The method of aspect 28, wherein the Cas nuclease is Cas9 or Cpf1.
30. The vector system of any of aspects 17-29, wherein the engineered retron comprises a CRISPR protospacer DNA sequence.
31. The vector system of aspect 30, further comprising a vector encoding a Cast or Cas2 protein.
32. The vector system of aspect 30 or 31, further comprising a vector comprising a CRISPR array sequence.
33. The vector system of any of aspects 29-31, further comprising a vector encoding bacteriophage homologous recombination proteins.
34. The vector system of aspect 33, wherein the vector encoding the bacteriophage homologous recombination proteins is a replication defective λ prophage comprising the exo, bet, and gam genes.
35. An isolated host cell comprising the engineered retron of any of aspects 1-16 or the vector system of any of aspects 17-34.
36. The host cell of aspect 35, wherein the host cell is a prokaryotic, archeon, or eukaryotic host cell.
37. The host cell of aspect 36, wherein the eukaryotic host cell is a mammalian host cell.
38. The host cell of aspect 37, wherein the mammalian host cell is a human host cell.
39 The host cell of aspect 35, wherein the host cell is an artificial cell or genetically modified cell.
40. A kit comprising the engineered retron of any of aspects 1-16, the vector system of any of aspects 17-34 or the host cell of any of aspects 35-39.
41. The kit of aspect 40, further comprising instructions for genetically modifying a cell with the engineered retron.
42. A method of genetically modifying a cell comprising:
   a) transfecting a cell with the engineered retron of aspect 1-15 or 16 (e.g., aspect 12);
   b) introducing or expressing an RNA-guided nuclease and a guide RNA into the cell, wherein the RNA-guided nuclease forms a complex with the guide RNA, said guide RNAs directing the complex to the genomic target locus, wherein the RNA-guided nuclease creates a double-stranded break in the genomic DNA at the genomic target locus, and the donor polynucleotide generated by the engineered retron is integrated at the genomic target locus recognized by its S′ homology arm and 3′ homology arm by homology directed repair (HDR) to produce a genetically modified cell.
43. The method of aspect 42, wherein the RNA-guided nuclease is a Cas nuclease or an engineered RNA-guided Fokl-nuclease.
44. The method of aspect 43, wherein the Cas nuclease is Cas9 or Cpf1.
45. The method of aspect 42-44, wherein the RNA-guided nuclease is provided by a vector or a recombinant polynucleotide integrated into the genome of the cell.
46. The method of aspect 42-45, wherein the engineered retron is provided by a vector.
47 The method of aspect 42-45 or 46, wherein the donor polynucleotide is used to create a gene replacement, gene knockout, deletion, insertion, inversion, or point mutation.
48. A method of genetically modifying a cell by recombineering, the method comprising:
   a) transfecting the cell with the engineered retron of aspect 1-16 (e.g., aspect 12); and
   b) introducing bacteriophage recombination proteins into the cell, wherein the bacteriophage recombination proteins mediate homologous recombination at a target locus such that the donor polynucleotide generated by the engineered retron is integrated at the target locus recognized by its 5′ homology arm and 3′ homology arm to produce a genetically modified cell.
49. The method of aspect 48, wherein the donor polynucleotide is used to modify a plasmid, bacterial artificial chromosome (BAC), or a bacterial chromosome in the bacterial cell by recombineering.
50. The method of aspect 48 or 49, wherein the donor polynucleotide is used to create a gene replacement, gene knockout, deletion, insertion, inversion, or point mutation.
51. The method of any of aspects 48-50, wherein said introducing bacteriophage recombination proteins into the cell comprises insertion of a replication-defective λ prophage into the bacterial genome.
52. The method of aspect 51, wherein the bacteriophage comprises exo, bet, and gam genes, 53. A method of barcoding a cell comprising transfecting a cell with the engineered retron of aspect 1-15 or 16 (e.g., aspect 15 or 16).
54. A method of producing an in vivo molecular recording system comprising:
   a) introducing a Cas1 protein or a Cas2 protein of a CRISPR adaptation system into a host cell;
   b) introducing a CRISPR array nucleic acid sequence comprising a leader sequence and at least one repeat sequence into the host cell, wherein the CRISPR array nucleic acid sequence is integrated into genomic DNA or a vector in the host cell; and
   c) introducing a plurality of engineered retrons according to aspect 1-16 (e.g., aspect 13 or 14) into the host cell, wherein each retron comprises a different protospacer DNA sequence that can be processed and inserted into the CRISPR array nucleic acid sequence.
55. The method of aspect 54, wherein the Cas1 protein or the Cas2 protein are provided by a vector.
56. The method of aspect 54 or 55, wherein the engineered retron is provided by a vector.
57. The method of any of aspects 54-56, wherein the plurality of engineered retrons comprises at least three different protospacer DNA sequences.
58. An engineered cell comprising an in vivo molecular recording system comprising:
   a) a Cas1 protein or a Cas2 protein of a CRISPR adaptation system;
   b) a CRISPR array nucleic acid sequence comprising a leader sequence and at least one repeat sequence into the host cell, wherein the CRISPR array nucleic acid sequence is integrated into genomic DNA or a vector in the engineered cell; and
   c) a plurality of engineered retrons according to aspect 1-16 (or aspect 13 or 14), wherein each retron comprises a different protospacer DNA sequence that can be processed and inserted into the CRISPR array nucleic acid sequence.
59. The engineered cell of aspect 58, wherein the Cas1 protein or the Cas2 protein are provided by a vector.
60. The engineered cell of aspect 58 or 59, wherein the engineered retron is provided by a vector.
61. The engineered cell of any of aspects 58-60, wherein the plurality of engineered retrons comprises at least three different protospacer DNA sequences.
62. A kit comprising the engineered cell of any of aspects 58-61 and instructions for in vivo molecular recording.
60. A method of producing recombinant msDNA comprising:
   a) transfecting a host cell with the engineered retron of any of aspects 1-16 or the vector system of any of aspects 17-34; and
   b) culturing the host cell under suitable conditions, wherein the msDNA is produced.

### EXAMPLES

The following examples are put forth so as to provide those of ordinary skill in the art with a disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees

Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Example 1: Materials and Methods

This Example illustrates some of the materials methods used in developing the invention.

### Bacterial Strains, Plasmids, and Culturing Conditions

Experiments were carried out in BL21-AI E. coli (Thermo Fisher), containing an integrated, arabinose-inducible T7 polymerase, an endogenous CRISPR array, an endogenous retron, but no endogenous Cas1+2 or bMS.346 a variant of MG1655 containing an integrated, arabinose-inducible T7 polymerase and no endogenous retron

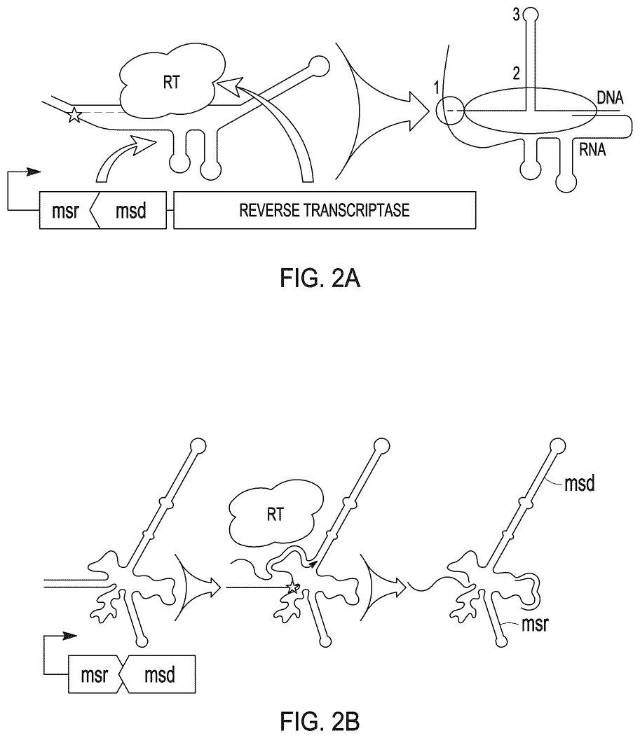

In the retron-based experiments depicted in FIGS. 3B, 3C, 4D, 7B, 7C-2, 8B, 9B, and 10B-D, the reverse transcriptase were expressed from a plasmid with an erythromycin-inducible promoter (mphR-ec86RT) (see Rogers et al., Nucleic Acids Res. 2015 Sep. 3;43 (15): 7648-60. doi: 10.1093/nar/gkv616. Epub 2015 Jul. 7 hereby incorporated by reference in its entirety.) The msd and msr elements were expressed from an inducible T7 promoter, either together (DUET-T7-msr/msd) or separately (DUET-T7-msr-T7-msd).

For the retron-generated protospacer experiments described with respect to FIGS. A-B, a plasmid encoding Cas1+2 and a modified ec86 msr/msd, both expressed by inducible (T7/lac) promoters (DUET-msr/msd-Cas1+2), was transformed into cells prior to each experiment. Cells containing plasmids were maintained in colonies on a plate at 4° C. for up to three weeks. Cells were grown in LB media at 34° C. and induced using IPTG, L-arabinose and/or erythromycin for the indicated durations.

### Electrophoretic Analysis of msd

To visualize the msd produced from modified retrons, bacteria were cultured for 4-16 hours in LB with all inducers necessary to express the msr-containing, msd-containing, and reverse-transcriptase-containing transcripts. A volume of 5-25 ml of culture was pelleted at 4° C., then prepared using a Plasmid Plus Midi Kit (Qiagen) or Mini Kit. The RNA was then digested using a combination of RNaseA and RNaseT1 and the resulting msd was purified using a ssDNA/RNA Clean & Concentrator kit (Zymo Research). The msd was visualized by running on a Novex TBE-Urea gel (Thermo Fisher) and post-staining with SYBR Gold (Thermo Fisher).

### Variant Library Construction

Retron ncRNA variant libraries were synthesized as oligo pool by Agilent or Twist with multiple libraries per synthesis run. Single libraries were amplified from these oligo pools and cloned into expression vectors using a golden gate approach, using NEB5a cells as the cloning strain. These cloned libraries were purified from the cloning strain and transferred into the expression strain (BL21-AI or bMS.346). All libraries were quantified by Illumina sequencing.

### Sequencing and Analysis

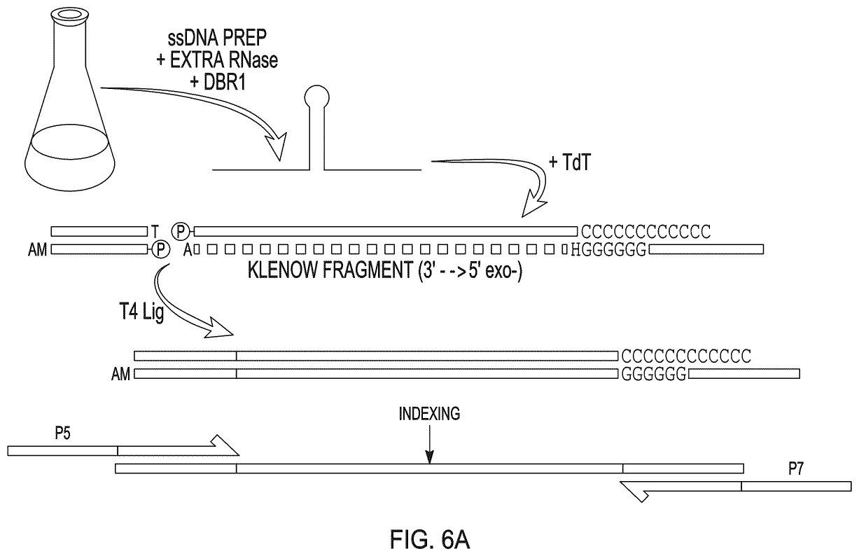

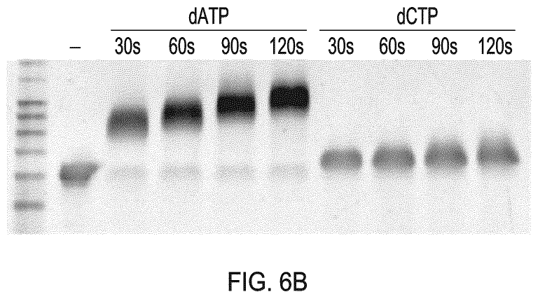

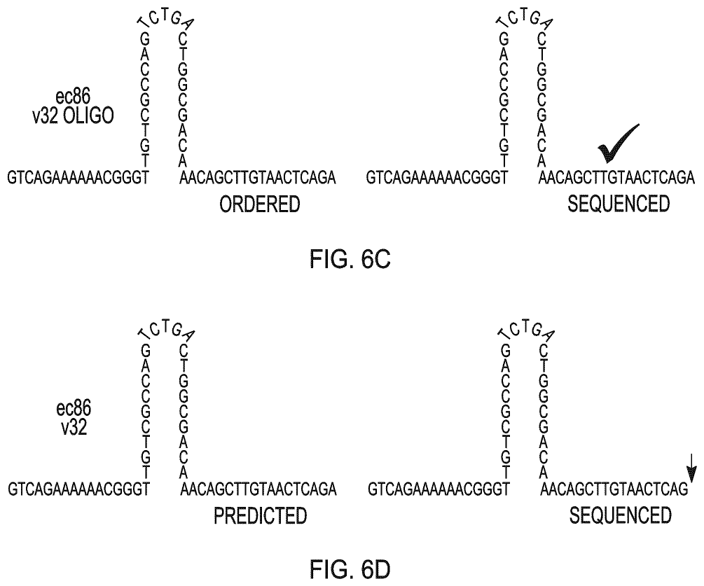

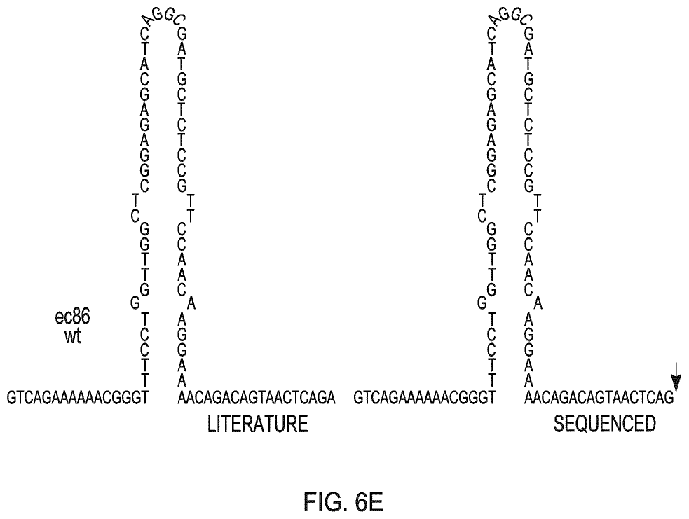

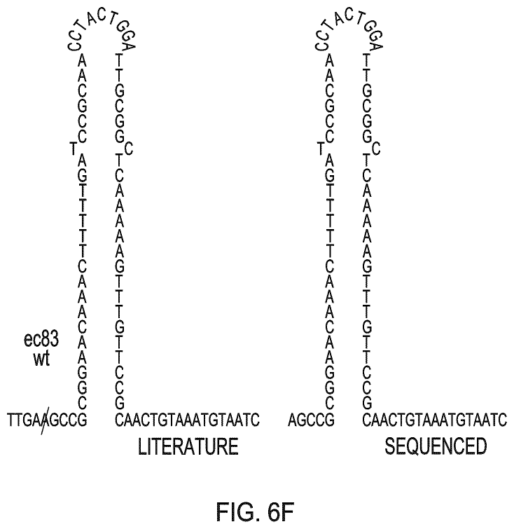

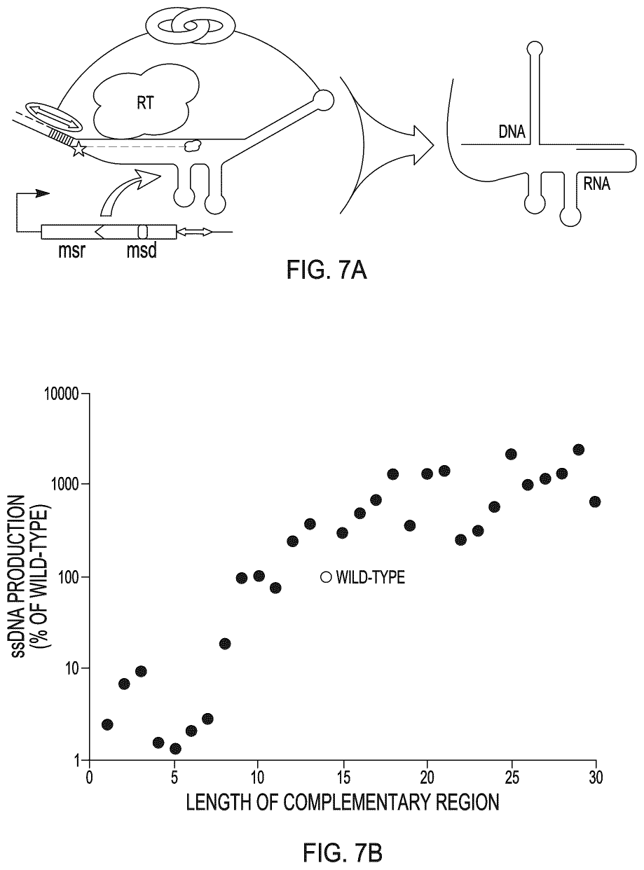

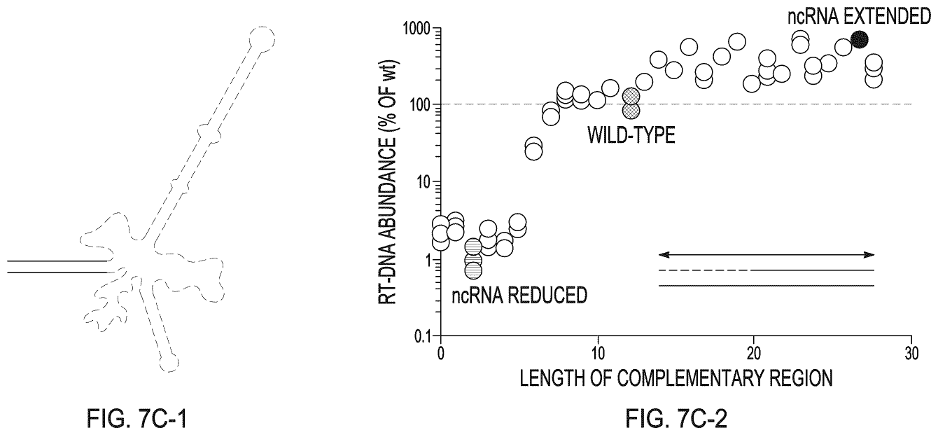

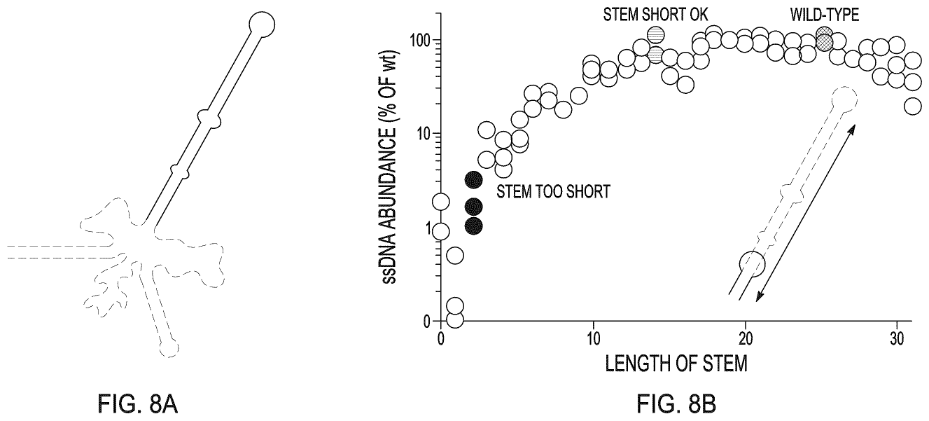

To quantify RT-DNA abundance in library experiments, reverse transcribed DNA was purified as described above after expression in cells containing a library of retron variants. A volume of 5-25 ml of culture was pelleted at 4° C., then prepared using a Plasmid Plus Midi Kit (Qiagen) or Mini Kit. The RNA was then digested using a combination of RNaseA and RNaseTI and the resulting msd was purified using a ssDNA/RNA Clean & Concentrator kit (Zymo Research). In variant libraries where the modifications were outside the reverse transcribed element (e.g. FIG. 7C-2), a barcoded region in the loop of the reverse transcribed DNA was amplified after expression and prepared for Illumina sequencing. In variant libraries where the modifications were inside the reverse transcribed element (e.g. FIG. 8B), the pool of purified reverse transcribed DNA was extended on the 3′ end with a single nucleotide using Terminal deoxynucleotidyl transferase (TdT), with the length of additional nucleotides controlled by the time of TdT incubation (FIG. 6B). Next, a reverse primer composed of an adapter sequence, a stretch of nucleotides complementary to the nucleotide used for extension, and an anchoring nucleotide (of every base that is not complementary to the nucleotide used for extension) was used to create a second strand using Klenow Fragment (3′→5′ exo-), which leaves an A overhang on the 5′ end. This overhanging A was used in a TA ligation to attach a double stranded adapter that was amino-modified on the opposing 5′ end (FIG. 6A). This pool of nucleotides with adapters added on both ends was then indexed and prepared for Illumina sequencing. In all variant libraries, the pool of plasmids present in the cells was quantified by amplifying the variable region and subjecting that region to Illumina sequencing. The relative abundance of different reverse transcribed DNAs was then calculated by comparing the ratio of the variant in the reverse transcribed DNA to the ratio of the variant plasmid, normalized to a co-expressed wild-type retron.

To analyze spacer acquisition, bacteria were lysed by heating to 95° C. for 5 minutes, then subjected to PCR of their genomic arrays using primers that flank the leader-repeat junction and additionally contain Illumina-compatible adapters. Spacer sequences were extracted bioinformatically based on the presence of flanking repeat sequences, and compared against pre-existing spacer sequences to determine the percentage of expanded arrays and the position and sequence of newly acquired spacers. New spacers were blasted (NCBI) against the genome and plasmid sequences and additionally compared against the intended protospacer sequence to determine the origin of the protospacer. This analysis was performed using custom written scripts in Python.

### Example 2: A Bacterial Retroelement (Retron) for Enhanced DNA Production in Cells

Rewriting the genome of living cells requires new DNA. Currently, workers synthesize this DNA exogenously and deliver it to cells, where it serves as a template for genome engineering or a barcode to mark cells or cellular events. However, it remains incredibly challenging to deliver exogenous DNA in enough abundance to overcome inefficiencies in the process of homology directed repair (HDR) and integration, particularly within complex tissues. Moreover, there is no way to gate the delivery by cell type or cell state to enable targeting subpopulations of cells with particular DNA sequences.

If we could produce designed DNA sequences in abundance and on demand inside cells of our choosing—as we do RNA and protein—we could overcome inefficiencies in HDR and unlock the ability to deliver different templates to different cells. We could use these locally produced DNA templates to shift from editing genomes to writing genomes. DNA on demand would fuel an array of DNA-modifying proteins, including λ Red Beta to recode bacterial genomes, Cas9 to write therapeutic modifications into the human genome, and the CRISPR integrases Cas1+2 to create molecular devices that log the timing of molecular events within living cells.

As described herein, reverse transcriptases are the solution to producing an abundance of DNA, including different DNAs with different sequences. Not only can they generate abundant DNA, but their activity can be controlled over time and space in the same way that we currently control RNA and protein expression. Thus, a broadly delivered reverse transcriptase can generate abundant template DNA in a targeted subset of cells.

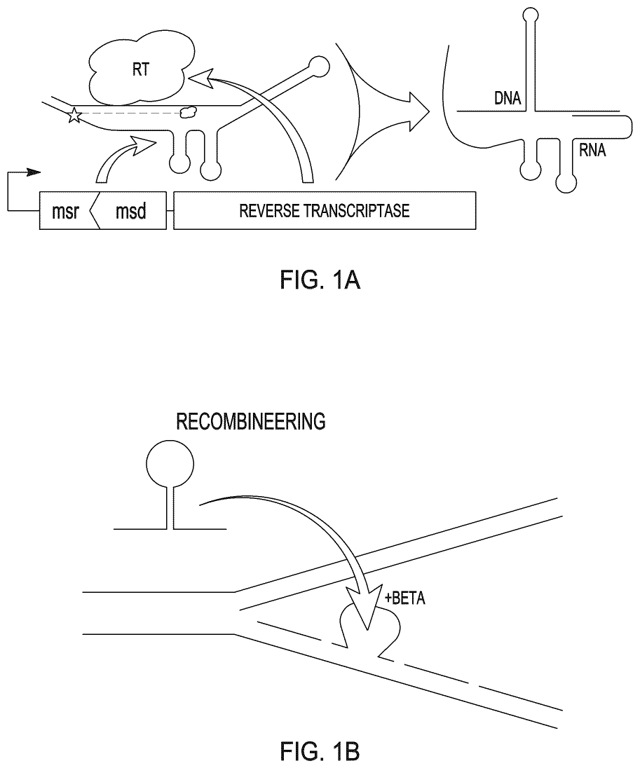

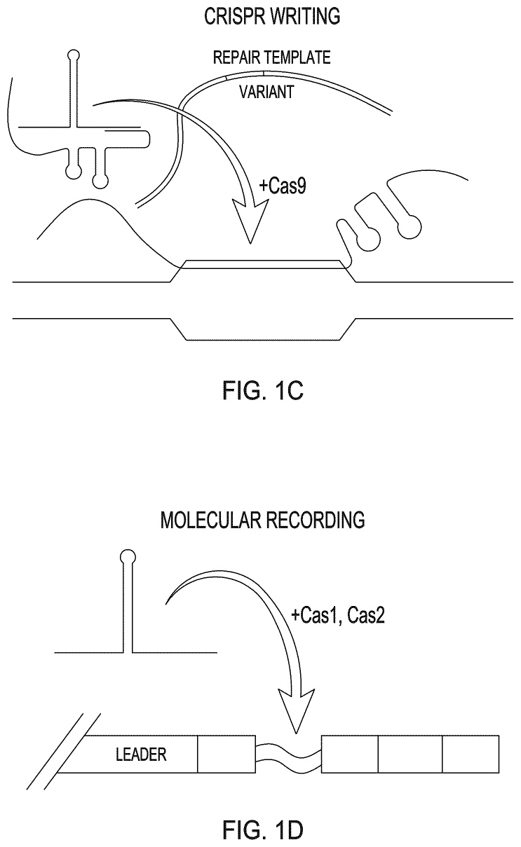

A particularly attractive class of reverse transcriptases come from bacteria and are termed retrons (Inouye & Inouye, Annual review of microbiology 45, 163-186 (1991)) (see, e.g. FIG. 1A). They are compact, modular, orthogonal to a eukaryotic cell, have been shown to produce DNA that is accessible to other proteins, and have served as template DNA for genome editing in both prokaryotic cells (Farzadfard & Lu Science 346, 1256272 (2014)) and eukaryotic cells (Sharon et al. Cell 175, 544-557 (2018)) (FIGS. 1B, 1C). Yet, there is still much we do not know about the biology of retrons and this knowledge gap prevents us from using retrons to generate completely designed sequences of DNA inside cells.

Here, we address the limitations of retrons directly by further characterizing and engineering the retron to produce CRISPR-compatible, arbitrary DNA sequences in high abundance within cells. The majority of the engineering was performed in E. coli, to achieve a high throughput, but the modified retrons and systems described herein can be used in eukaryotic cells (including human) to provide improvements in the context of genome writing.

For example, in some cases retron-Ecol was used as an exemplary retron. This transcript is recognized by the reverse transcriptase and is partially reverse transcribed into RT-DNA, as shown in FIG. 2A. The sequence of the reverse-transcribed retron-Ecol DNA (RT-DNA) is shown below as SEQ ID NO: 14.

| | |
|---|---|
| 1 | GTCAGAAAAA ACGGGTTTCC TGGTTGGCTC GGAGAGCATC |
| 41 | AGGCGATGCT CTCCGTTCCA ACAAGGAAAA CAGACAGTAA |
| 81 | CTCAGA |

### Example 3: Screening Engineered Retron Variants

The inventors expressed the retron-Eco1 (also called ec86 or retron-Eco1 ncRNA) in E. coli. The sequence for this wild type retron-Eco1 ncRNA is shown below as SEQ ID NO: 15.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGGAGAGCA TCGCCTGATG |
| 121 | CTCTCCGAGC CAACCAGGAA ACCCGTTTTT TCTGACGTAA |
| 161 | GGGTGCGCA |

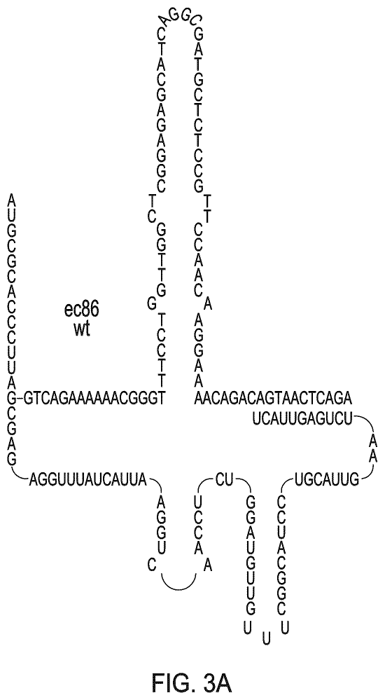

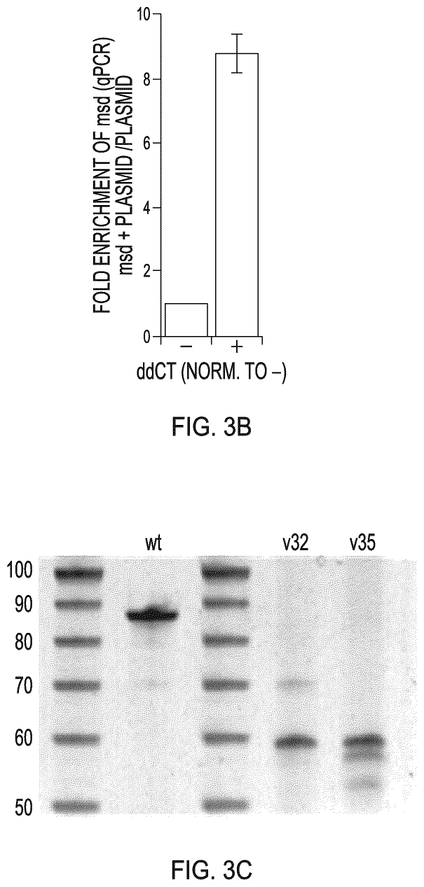

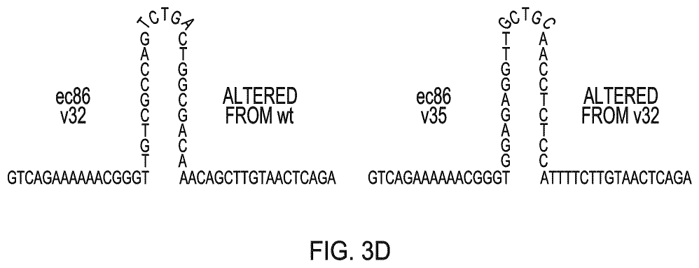

Quantitative PCR (qPCR) showed that expression of expressed the retron-Eco1 in E. coli yielded about 800-1,000 copies of ssDNA per cell (FIGS. 3B). As illustrated in FIG. 3C, the ssDNA so produced can be visualized, quantified and purified on a denaturing gel.

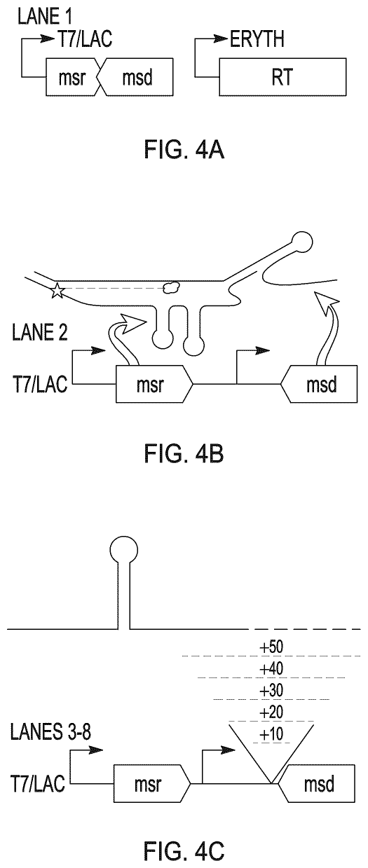

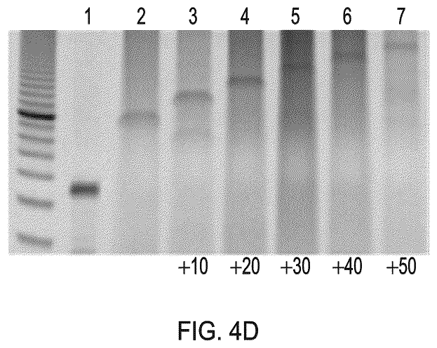

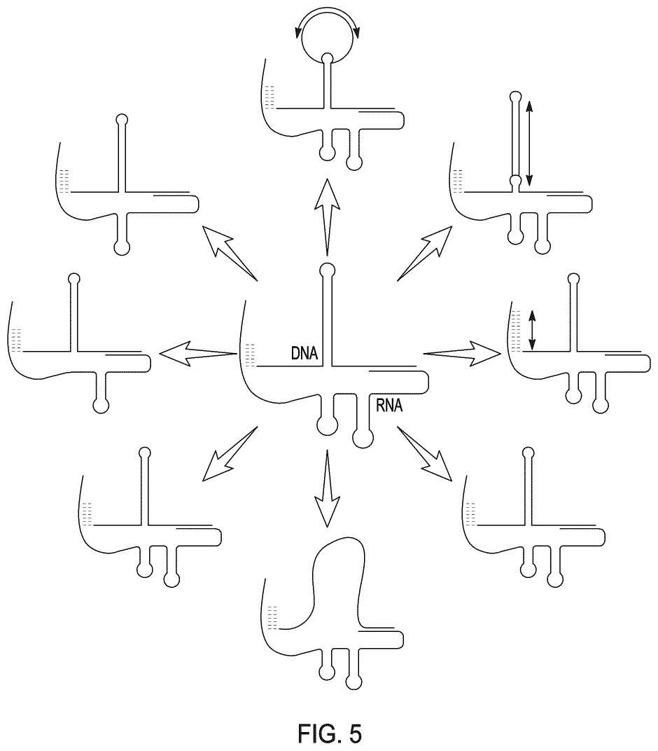

The inventors also made constructs encoding various retron elements. For example, the reverse transcriptase was separated the from the msr/msd (primer-template), allowing the msr and msd to be supplied in trans to the reverse transcriptase (rather than in the typical cis arrangement) (FIGS. 4A, 4B). This trans arrangement eliminates a cryptic stop signal for the reverse transcriptase. The sequence of the retron-Eco1 msr only region is shown below as SEQ ID NO: 16.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | AAGCTGTTTG TCGCCAG |

The sequence of the retron-Eco1 msd only region is shown below as SEQ ID NO:17.

| | |
|---|---|
| 1 | AATCTGAGTT ACAAGCTGTT TGTCGCCAGT CAGACTGGCG |
| 41 | ACAACCCGTT TTTTCTGACG TAAGGGTGCG CA |

Using low throughput experiments, changes were made to the msd element that were tolerated by the reverse transcriptase. For example, two variants were produced, a retron-Eco1 v32 ncRNA and retron-Eco1 v35 ncRNA. The sequence of the retron-Eco1 v32 ncRNA is shown below as SEQ ID NO: 18.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | AAGCTGTTTG TCGCCAGTCA GACTGGCGAC AACCCGTTTT |
| 121 | TTCTGACGTA AGGGTGCGCA |

The sequence of the retron-Eco1 v35 ncRNA is shown below as SEQ ID NO:19.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | AAGCTGTTTG TCGCCAGTCA GACTGGCGAC AACCCGTTTT |
| 121 | TTCTGACGTA AGGGTGCGCA |

Key sections of the retron-Eco1 v32 ncRNA and retron-Eco1 v35 ncRNA are shown in FIG. 3D.

Numerous modifications were made to the retron-Eco1 ncR.NA. However, not every attempted modification has been successful. In some cases, the majority of modified versions produced no ssDNA in cells.

To better understand the determinants of msd production from the retron, the inventors have taken a library-based approach. Tens of thousands of retron variants were synthesized to systematically test each structural parameter of the retron (FIG. 5). A golden-gate-based cloning strategy (Engler et al., PLOS One (Nov. 5, 2008) was used to clone these variants, and then large pools of modified retrons were expressed in a multiplexed experiment along with the reverse transcriptase. By purifying all msds produced by these cells, sequencing them, and comparing their abundance to that of their retron/plasmid of origin in the expression strain, the influence of particular parameters of the retron was quantified as it related to ssDNA production. The ec86 retron was used as well as other retrons, including the ec83 retron, which has an internal branch structure.

The retron reverse transcriptase typically primes in a non-standard manner to create a branched RNA-DNA hybrid, linking the msr RNA at a 2′ position to the 5′ end of the msd ssDNA via a phosphodiester bond (Inouye & Inouye, Annual review of microbiology 45, 163-186 (1991)). E. coli have no enzyme to cleave this bond. Hence, when within E. coli the ec86 retron remains branched. However, ec83 has also been reported to be processed through an unknown mechanism that is intrinsic to the retron, eliminating the 2′-5′ linkage and freeing the ssDNA (Lim, Molecular microbiology 6, 3531-3542 (1992)). Such a separation may benefit various applications in genome engineering.

### Example 4: Sequencing Engineered Retron Variants

Sequencing the retron-derived ssDNA as a read-out of the experiment introduces significant complexity, as the pool of purified ssDNA contains unknown portions (e.g. different ends), by design. These ssDNA cannot be prepared for multiplexed sequencing using traditional pipelines. To address this challenge, the inventors have developed a custom sequencing pipeline, which involves purifying the ssDNA, treating the ssDNA with RNAase, and debranching the retron-derived ssDNA. The purified and debranched retron ssDNAs were then tailed with a string of polynucleotides of a single type using a template independent polymerase (TdT). Complementary strands of the ssDNAs were then generated using an adapter-containing, inverse anchored primer (FIGS. 6A, 6B). To this double-stranded DNA, a second adapter is ligated, and this adapter-linked, double-stranded DNA is then indexed and subjected to multiplexed sequencing.

This pipeline has been validated using synthesized oligonucleotides, wild-type and modified ec86, and wild-type ec83. The method reliably determines the correct sequence of a synthesized oligonucleotide.

Interestingly, using this multiplexed, single molecule method, the ec86 retron-derived ssDNAs typically terminate one base earlier than was reported in the literature using older, bulk methodologies (e.g. Maxam-Gilbert sequencing). The cleavage and predicted endogenous exonuclease processing of ec83 was also confirmed (FIGS. 6C-6F). Because retron-derived ssDNA are sequenced, modifications of the msd (template) part of the retron can be read directly.

The inventors also aim to understand the parameters of the neRNA that are not reverse transcribed. To read out these parameters, variants in the non-reverse transcribed region were linked to barcodes inserted in the loop region of the msd (FIGS. 7A). This approach illuminated the effect of sequence variations, e.g., on ssDNA production, even though the variants were not sequenced directly.

### Example 5: Modifications that Increase the Production of DNA in Cells

This Example illustrates that separation of the msr and msd transcripts can allow for the production of longer RT-DNA and that modification of the length of retron self-complementary, non-coding RNA regions can increase the abundance of reverse transcribed DNA generated by the retron.

Expression of the trans constructs encoding the retron-Eco1 msr and msd elements as described in Example 3 eliminated a cryptic stop signal for the reverse transcriptase and allowed the generation of longer ssDNAs to be generated (FIGS. 4C, 4D).

One example of an extended trans retron-Eco1 msd sequence is referred to as retron-Eco1 msd +50 and is shown below as SEQ ID NO:20.

| | |
|---|---|
| 1 | TGGACAATAT TGAATGGAGT CTGATCAACC TTCACACCGA |
| 41 | TCTAGAATCG GAATCTGAGT TACAAGCTGT TTGTCGCCAG |
| 81 | TCAGACTGGC GACAACCCGT TTTTTCTGAC GTAAGGGTGC |
| 121 | GCA |

As shown in FIG. 7, extension of a self-complementary region at the 5′ and 3′ ends of the retron-Eco1 ncRNA leads to a large increase in the abundance of RT-DNA produced in cells by the retron. One example of an extended retron-Eco1 ncRNA is shown below as SEQ ID NO:21.

| | |
|---|---|
| 1 | TGATAAGATT CCGTATGCGC ACCCTTAGCG AGAGGTTTAT |
| 41 | CATTAAGGTC AACCTCTGGA TGTTGTTTCG GCATCCTGCA |
| 81 | TTGAATCTGA GTTACTGTCT GTTTTCCTTG TTGGAACGGA |
| 121 | GAGCATCGCC TGATGCTCTC CGAGCCAACC AGGAAACCCG |
| 161 | TTTTTTCTGA CGTAAGGGTG CGCATACGGA ATCTTATCA |

An example of an extended retron-Eco1 v35 ncRNA with a somewhat different sequence is shown below (SEQ ID NO:22).

| | |
|---|---|
| 1 | TGATAAGATT CCGTATGCGC ACCCTTAGCG AGAGGTTTAT |
| 41 | CATTAAGGTC AACCTCTGGA TGTTGTTTCG GCATCCTGCA |
| 81 | TTGAATCTGA GTTACAAGCT GTTTGTCGCC AGTCAGACTG |
| 121 | GCGACAACCC GTTTTTTCTG ACGTAAGGGT GCGCATACGG |
| 161 | AATCTTATCA |

As illustrated in FIGS. 7B-7C-2, extension of a self-complementary region at the 5′ and 3′ ends of the retron-Eco1 neRNA leads to a large increase in the abundance of RT-DNA produced in cells by the retron. For example, a 10-fold increase in the relative amount of ssDNA can be produced by increasing the length of the msd sequence self-complementary region.

Conversely, reduction in the ncRNA self-complementary bases greatly diminished the production of RT-DNA. For example, one sequence for a retron-Eco1 ncRNA with a shorter self-complementary sequence is shown below as SEQ ID NO: 23.

| | |
|---|---|
| 1 | TAGCGAGAGG TTTATCATTA AGGTCAACCT CTGGATGTTG |
| 41 | TTTCGGCATC CTGCATTGAA TCTGAGTTAC TGTCTGTTTT |
| 81 | CCTTGTTGGA ACGGAGAGCA TCGCCTGATG CTCTCCGAGC |
| 121 | CAACCAGGAA ACCCGTTTTT TCTGACGTA |

As shown in FIGS. 7C-1 and 7C-2, reduction in the numbers of these complementary bases greatly diminished the production of RT-DNA.

Therefore, extension of pre-msr/post-msd self-complementarity region can increase the pool of ssDNAs. The larger pool of reverse transcribe ssDNAs can be available for genetic modification and can increase the efficiency of genome editing in bacteria (recombineering), yeast (CRISPEY), and mammalian cells. For the purpose of producing abundant DNA in living cells, these variants with extended self-complementary regions are preferred.

### Example 6: Msd Stem Region Tolerates Some. Not All. Modifications

Modifications were made to the msd stem region of the retron-Eco1 ncRNA region to disrupt the stem secondary structure (double-stranded bonding). This Example illustrates where modifications can be made to the msd stem without adversely affecting the abundance of reverse transcribed ssDNA produced by the retron.

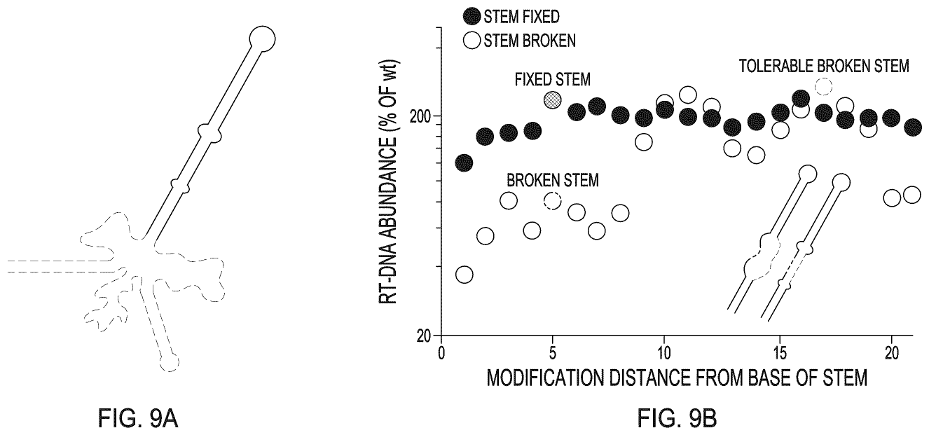

The positions modified along the msd stem are illustrated in FIGS. 8A-8B and 9A-9B.

Modifications to the length of the msd stem structure can create shorter sequences of RT-DNA. For example, one sequence for a retron-Eco1 ncDNA with a short stem short that still provides wild type levels of ssDNA (retron-Eco1 stem short OK, see FIG. 8B) is shown below as SEQ ID NO:24.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA AGCCTAGCCA ACCAGGAAAC |
| 121 | CCGTTTTTTC TGACGTAAGG GTGCGCA |

However, as the msd stem length decreases below 14 bases, the abundance of RT-DNA produced is negatively affected (FIG. 8B). One example of a sequence retron-Eco1 ncDNA with a stem that is too short is shown below as SEQ ID NO:25 (FIG. 8B, stem too short).

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTG CCTAACCCGT TTTTTCTGAC GTAAGGGTGC |
| 121 | GCA |

In contrast, the amount of ssDNA generated is the same or somewhat higher than wild type when the stem region of a retron is broken and then repaired. By “broken” is meant that the base-pairing of the stem is undermined, for example, by introducing non-complementary nucleotides. As illustrated by FIG. 9B, when the base of the ncRNA stem is broken by changing five bases in a row the abundance of RT-DNA is drastically reduced. A sequence for such a retron-Eco1 ncRNA with five mismatched bases (broken stem, FIG. 9B) is shown below as SEQ ID NO: 26.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CGAACATGGA ACGGAGAGCA TCGCCTGATG |
| 121 | CTCTCCGAGC CAACCAGGAA ACCCGTTTTT TCTGACGTAA |
| 161 | GGGTGCGCA |

However, if those bases are compensated by complementary changes on the other side of the stem to preserve the stem secondary structure, the RT-DNA abundance is preserved (see e.g., fixed stem, FIG. 9B). A sequence for the ‘fixed stem’ retron-Eco1 ncRNA shown in FIG. 9B is shown below as SEQ ID NO: 27.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CGAACATGGA ACGGAGAGCA TCGCCTGATG |
| 121 | CTCTCCGAGC CATGGTCGAA ACCCGTTTTT TCTGACGTAA |
| 141 | GGGTGCGCA |

Modifications to the stem in a region that is 9 to 20 bases from the base of the stem are tolerated even if they break the stem structure (FIG. 9B, see the tolerable broken stem). Such a tolerable broken stem has modifications (mismatches) to the middle of the stem. One example of a sequence for a retron-Eco1 ncRNA with the tolerable broken stem of FIG. 9B is shown below as SEQ ID NO:28.

| | |
|---|---|
| 1 | TGCGCACCCT TAGCGAGAGG TTTATCATTA AGGTCAACCT |
| 41 | CTGGATGTTG TTTCGGCATC CTGCATTGAA TCTGAGTTAC |
| 81 | TGTCTGTTTT CCTTGTTGGA ACGCTCTCCA TCGCCTGATG |
| 121 | CTCTCCGAGC CAACCAGGAA ACCCGTTTTT TCTGACGTAA |
| 161 | GGGTGCGCA |

Hence, for the purpose of producing DNA in living cells, the sequence of the ncRNA can be modified as long as the base of the stem structure is preserved.

Modifications to middle of the msd stem, about 9-20 bases from the base of the stem, are tolerated and do not adversely affect reverse transcription of ssDNA.

### Example 7: The ncRNA msd Stem Region Center Is More Tolerant of Modifications

Small modifications were made to various positions within the region of the retron-Eco1 ncRNA region that is reversed transcribed (msd) and the impact of those modifications was measured on the amounts of ssDNA reversed transcribed from the different neRNA variants.

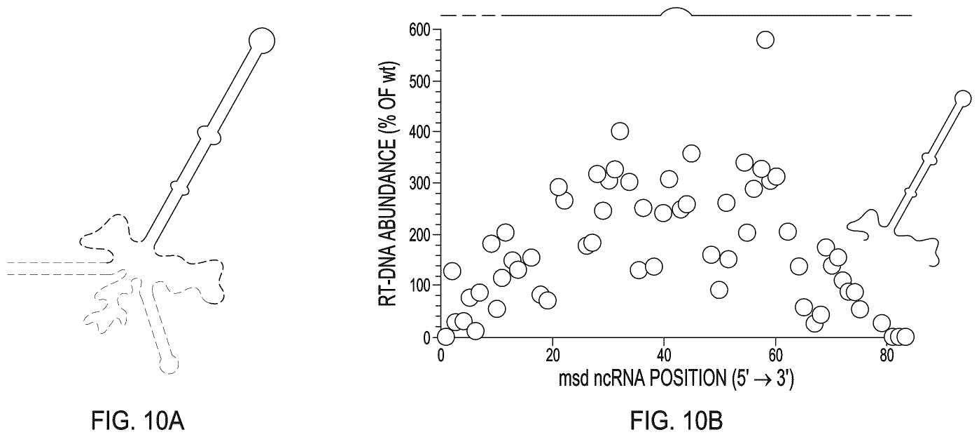

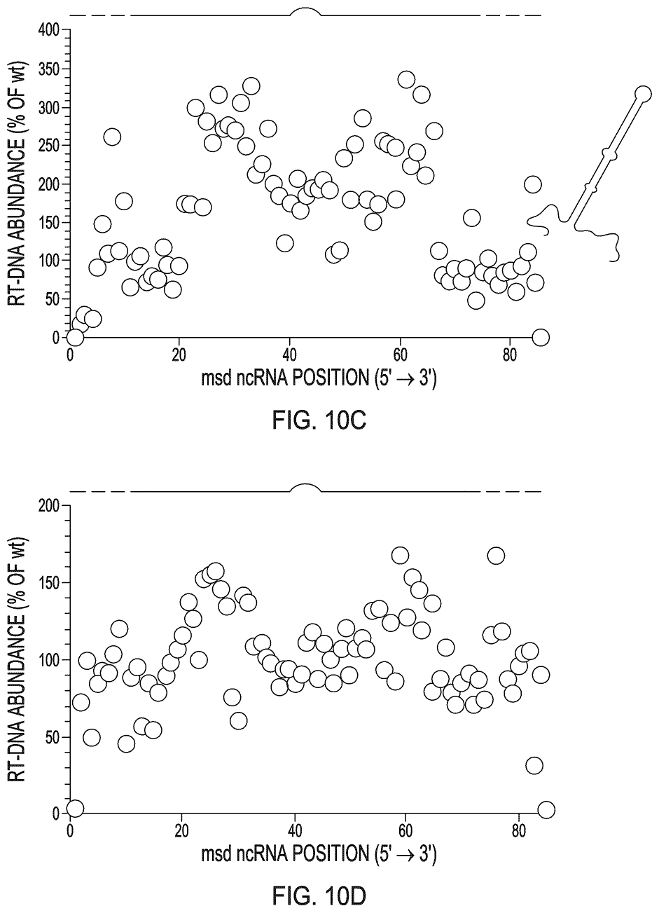

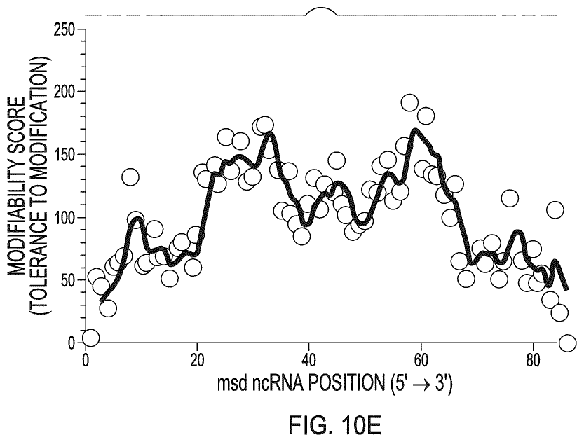

Tolerance to small modifications throughout the reverse transcribed region of the ncRNA are variable as illustrated in FIG. 10. FIG. 10B shows the effects of deleting three bases from various positions along the msd complementary (stem) region. As shown, deletion of three bases from the middle of the msd stern had no adverse effects and still led to high levels of ssDNA production (FIG. 10B). However, lower levels of ssDNA production were observed when deletions of three bases were made nearer the base of the complementary (stem) region or in the regions flanking the stem (FIG. 10B). Similar effects were observed for insertions of three bases (FIG. 10C), and single base changes (FIG. 10D) in the middle and flanking parts of the msd region. While the middle of the msd stem region tolerated insertions and/or deletions of several nucleotides (e.g. of less than 5 nucleotides) such that no significant reduction in ssDNA production was observed, such modifications to the flanking sequences at the base of the msd stem were not tolerated. Modification of the base and flanking regions of the msd stem led to reduced reverse transcription of ssDNA.

FIG. 10E graphically illustrates a modifiability score for positions within the msd stem region that was calculated based on the data in FIG. 10B-10D. For the purpose of producing higher levels of DNA in living cells, the sequence of the ncRNA should be modified in regions with a high modifiability score and modification of regions with low modifiability scores should be avoided.

### Example 8: Applications of Engineered Retrons

Creating DNA on demand in cells with engineered retrons enables to shift from editing genomes in living cells to writing genomes. This shift will let us therapeutically modify cells without being restricted by previously existing sequences. Currently, new DNA HDR templates are necessarily delivered as a bolus that declines over time. The inefficiencies with this bolus delivery mean that they cannot be written in situ, but instead must be written in vitro, followed by selection and expansion. Not all experiments, and few therapeutics, are compatible with this strategy.

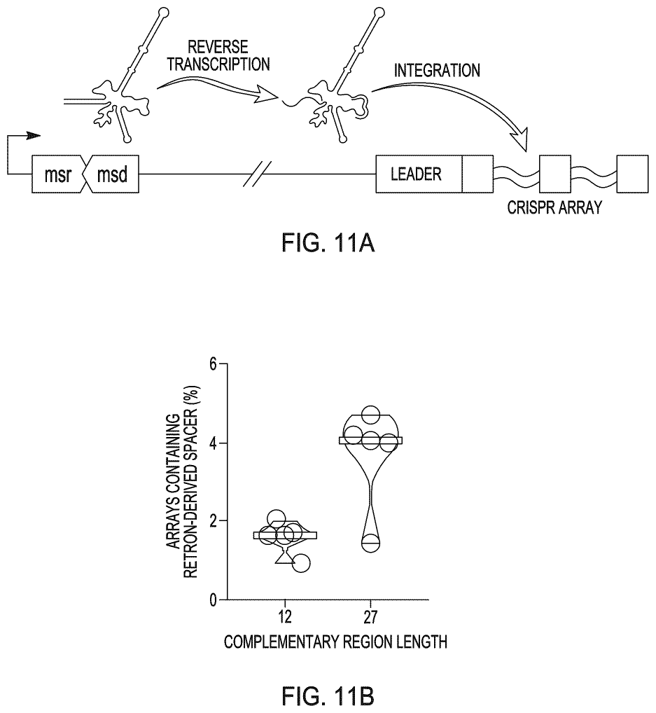

To fully realize the potential of CRISPR-based therapeutics, the inventors provide designed sequences of DNA to rewrite the genome exactly when and where they are needed. The designed sequences can be provided as illustrated in FIG. 11A. The efficiency of modification is increased by extending the region of self-complementary at the 5′ and 3′ ends of the ncRNA and the retron reverse transcriptase can be mobilized to produce an abundance of the desired ssDNA therefrom.

Producing DNA on demand also enables a novel application of genome engineering, aimed not at therapeutics directly, but rather at understanding the biology of disease. This is the field of molecular recording, where modifications to a cell's genome are used to write data within a living system. See, e.g., Shipman et al. (2017) Nature 547(7663): 345-349, and Shipman et al. (2016) Science 353(6298): aaf1175 for a description of this approach using the CRISPR integrases Cas1+2 to capture short sequences of DNA in a CRISPR array, logged in order over time to store a record of events; herein incorporated by reference in their entireties. This approach can be used as the back end of a data acquisition device in cells. However, it requires a front end that generates DNA barcodes in cells, driven by biological events like transcription. Retron-derived DNA can enable these types of technologies and allow us to understand complex biological processes with a level of detail that has never before been achieved.

Ultimately, domesticating the retron expands our repertoire of molecular biotechnology aimed at genome engineering. Retrons can be designed with modular components to make arbitrary DNA sequences in living cells on demand, with implications extending broadly to scientists interested in genetic therapies, cellular control, and cell engineering.

### REFERENCES

1 Inouye, M. & Inouye, S. msDNA and bacterial reverse transcriptase. Annual review of microbiology 45, 163-186, doi: 10.1146/annurev.mi.45.100191.001115 (1991).
2 Farzadfard, F. & Lu, T. K. Synthetic biology. Genomically encoded analog memory with precise in vivo DNA writing in living cell populations. Science 346, 1256272, doi: 10.1126/science. 1256272 (2014).
3 Sharon, E. et al. Functional Genetic Variants Revealed by Massively Parallel Precise Genome Editing. Cell 175, 544-557. e516, doi: 10.1016/j.cell.2018.08.057 (2018).
4 Dhundale, A. R., Furuichi, T., Inouye, S. & Inouye, M. Distribution of multicopy single-stranded DNA among myxobacteria and related species. Journal of bacteriology 164, 914-917 (1985).
5 Lampson, B. C. et al. Reverse transcriptase in a clinical strain of Escherichia coli: production of branched RNA-linked msDNA. Science 243, 1033-1038(1989).
6 Inouye, K., Tanimoto, S., Kamimoto, M., Shimamoto, T. & Shimamoto, T. Two novel retron elements are replaced with retron-Vc95 in Vibrio cholerae. Microbiology and immunology 55, 510-513, doi: 10.1111/j. 1348-0421.2011.00342. x (2011).
7 Lim, D. Structure and biosynthesis of unbranched multicopy single-stranded DNA by reverse transcriptase in a clinical Escherichia coli isolate. Molecular microbiology 6, 3531-3542 (1992).
8 Shipman, S. L., Nivala, J., Macklis, J. D. & Church, G. CRISPR-Cas encoding of a digital movie into the genomes of a population of living bacteria. Nature, doi: 10.1038/nature23017 (2017).
9 Shipman, S. L., Nivala, J., Macklis, J. D. & Church, G. M. Molecular recordings by directed CRISPR spacer acquisition. Science, doi: 10.1126/science.aaf1175 (2016).

While the present disclosure has been described with reference to the specific embodiments thereof, it should be understood by those skilled in the art that various changes may be made and equivalents may be substituted without departing from the true spirit and scope of the present disclosure. In addition, many modifications may be made to adapt a particular situation, material, composition of matter, process, process step or steps, to the objective, spirit and scope of the present disclosure. All such modifications are intended to be within the scope of the claims appended hereto.

1. (canceled)

2. An engineered retron comprising:
a) a pre-msr sequence;
b) an msr gene encoding multicopy single-stranded RNA (msRNA);
c) an msd gene encoding multicopy single-stranded DNA (msDNA); and
d) a post-msd sequence comprising a self-complementary region having sequence complementarity to the pre-msr sequence,
e) a ret gene encoding a reverse transcriptase,
wherein the msr gene and the msd gene are provided in a trans arrangement or wherein the ret gene is in a trans arrangement with respect to the msr gene or the msd gene,
wherein the msd gene or the msr gene comprises a heterologous sequence,
wherein the heterologous sequence encodes a donor polynucleotide comprising a 5′ homology arm that hybridizes to a 5′ target sequence and a 3′ homology arm that hybridizes to a 3′ target sequence flanking a nucleotide sequence comprising an intended edit to be integrated at a target locus by homology directed repair (HDR) or recombineering.

3. The engineered retron of claim 2, wherein the ret gene is trans to the msr gene and msd gene.

4. The engineered retron of claim 2, wherein the msr gene and msd gene are cis to each other and trans to the ret gene.

5. The engineered retron of claim 2, wherein the cryptic stop signal is removed from the ret gene.

6. A vector system comprising one or more vectors comprising the engineered retron of claim 2.

7. The vector system of claim 6, wherein the msr gene and the msd gene are provided by the same vector or different vectors.

8. The vector system of claim 6, wherein the ret gene is provided by a vector different than the msr and msd genes.

9. The vector system of claim 6, wherein the same vector comprises a promoter operably linked to the msr gene and the msd gene.

10. The vector system of claim 9, further comprising a second promoter operably linked to the ret gene.

11. The vector system of claim 6, wherein the one or more vectors are viral vectors or nonviral vectors.

12. The vector system of claim 6, wherein the nonviral vectors are plasmids.

13. The vector system of claim 6, further comprising a vector encoding an RNA-guided nuclease.

14. The vector system of claim 13, wherein the RNA-guided nuclease is a Cas nuclease or an engineered RNA-guided FokI-nuclease.

15. The vector system of claim 14, wherein the Cas nuclease is Cas9 or Cpf1.

16. An isolated host cell comprising the engineered retron of claim 2 or the vector system of any of claim 6.

17. The host cell of claim 16, wherein the host cell is a prokaryotic, archeon, or eukaryotic host cell.

18. A kit comprising the engineered retron of claim 2, the vector system of claim 5, or the host cell of claim 17.

19. A method of genetically modifying a cell comprising:
a) transfecting a cell with the engineered retron of claim 2;
b) introducing an RNA-guided nuclease and guide RNA into the cell, wherein the RNA-guided nuclease forms a complex with the guide RNA, said guide RNAs directing the complex to the genomic target locus, wherein the RNA-guided nuclease creates a double-stranded break in the genomic DNA at the genomic target locus, and the donor polynucleotide generated by the engineered retron is integrated at the genomic target locus recognized by its 5′ homology arm and 3′ homology arm by homology directed repair (HDR) to produce a genetically modified cell.

20. A method of producing recombinant msDNA comprising:
a) transfecting a host cell with the engineered retron of claim 2 or the vector system of claim 6; and
b) culturing the host cell under suitable conditions, wherein the msDNA is produced.
